(19) United States
(12) Patent Application Publication
(10) Pub. No.: US 2010/0145301 A1
(43) Pub. Date: Jun. 10, 2010

(54) SPRAY ADMINISTRATION OF COMPOSITIONS INCLUDING ACTIVE AGENTS SUCH AS PEPTIDES TO THE GASTROINTESTINAL TRACT

(75) Inventor: Elad Magal, Ramat-HaSharon (IL)

Correspondence Address: HESLIN ROTHENBERG FARLEY & MESITI PC, 5 COLUMBIA CIRCLE, ALBANY, NY, 12203, US

(73) Assignee: DUOCURE, INC., Ramat-HaSharon (IL)

(21) Appl. No.: 12/522,267

(22) PCT Filed: Feb. 7, 2008
(86) PCT No.: PCT/IL2008/000170

§ 371 Date: Jan. 19, 2010

Related U.S. Application Data
(60) Provisional application No. 60/903,289, filed on Feb. 26, 2007.

(51) Int. Cl. A61M 5/142; A61K 38/22

(52) U.S. Cl. 604/503; 514/16; 604/131; 604/500

(57) ABSTRACT

Methods of administering an active agent such as an active pharmaceutical ingredient by spraying a composition comprising the active agent at a luminal wall of the gastrointestinal tract are disclosed. Also disclosed are devices for administering a composition suitable for implementing the disclosed method. Also disclosed is the use of a peptide as an active agent for the manufacture of a sprayable composition for use in the treatment of a subject by gastrointestinal administration.

### FIELD AND BACKGROUND OF THE INVENTION

The present invention relates to administration of active agents such as active pharmaceutical ingredients and more particularly to the administration of active agents by spraying a sprayable composition including the active agent at a portion of the wall of the lumen of the gastrointestinal tract.

In the medical and health care arts there is a continuous search for methods of effective administration of active agents, whether by different routes (e.g., topical, oral, transdermal, subcutaneous, intravenous) or different types of compositions (e.g. liquids, gels, tablets, foams). One logical method used for thousands of years is through the gastrointestinal tract: a composition containing the active agent is orally ingested and passes through the gastrointestinal tract, during which passage the active agent acts, for example topically by treating a pathology on the lumen of the gastrointestinal tract or by stimulating sites such as chemoreceptors on the lumen of the gastrointestinal tract or for example, systemically by absorption through the walls of the gastrointestinal tract to enter the circulatory system.

Administration of active agents through the gastrointestinal tract is inefficient due to many factors including the varying chemical and physical environmental conditions along the gastrointestinal tract, the fact that most active agents act or are absorbed only at specific locations of the gastrointestinal tract and the fact that many active agents are susceptible to degradation of one or more organs of the gastrointestinal tract. As a result, efforts have been made to provide compositions that deliver an effective dose of an active agent to a specific location of the gastrointestinal tract. Such compositions include delayed-release compositions which release an active agent a predetermined time after ingestion or targeted-release compositions which release an active agent only upon exposure to conditions present in a specific location of the gastrointestinal tract.

An alternative approach is taught in PCT patent application PCT/IL2005/001053 published as WO 2006/035446 of the Applicant in the context of treating conditions relating to overeating such as obesity. A central concept taught therein is the delivery of a beneficial stimulus at the “right time” (only when needed) at the “right place” (to a localized part of the body where most effective) which allows the “right dose” to be administered (no over- or under-dosing). Such an approach has the potential of increased efficacy, with less stimulation, fewer side effects and reduced chance for sensitization.

The specific teachings of WO 2006/035446 are based on the fact that the desire to eat is, in a large part, driven by hunger which stops upon the perception of satiation. Various mechanoreceptors and chemoreceptors in the gastrointestinal tract detect the degree of distension of the gastrointestinal tract and release satiety factors. Known satiety factors that are released to control food ingestion include Cholecystokinin (CCK), Bombesin, Gastrin-releasing peptide (GRP), Glucagon, Glucagon-like peptide (GLP-1), Enterostatin and Ghrelin. As there is a delay between the time of food ingestion and the release of the satiety factors, it is common for a person to overeat. Apart from the direct weight gain caused by consuming too much food, overeating also causes the base volume of the stomach to increase and the gastric mechanoreceptors to become insensitive to small increases of stomach volume. Thus, a positive-feedback loop with negative consequences is generated where a person inherently overeats as indication of satiety occurs only after satiety is reached, so that the person overeats, reducing the sensitivity of the satiety sensors, so that the indication of satiety is delayed even further.

To resolve this problem, WO 2006/035446 teaches a device capable of sensing a physiological change associated with food ingestion or hunger (i.e., identifying the “right time”) and a mechanism adapted for directly stimulating a region of the body responsive to a gastrointestinal satiety agent which is implanted in the body (i.e., the “right place”). Specific mechanisms for sensing a physiological change associated with food ingestion or hunger include muscle activity sensors and pressure sensors. Specific mechanisms adapted for stimulation include drug dispensers, space-filling balloons, vagal-mechanoreceptor stimulating balloons and nerve-stimulating electrodes. Upon detection of a physiological change associated with food ingestion or hunger, the stimulation mechanism is activated giving the person a feeling of satiety. By detecting that a person is about to eat or has started eating before having consumed too much food and then stimulating a feeling of satiety, the teachings of WO 2006/035446 treat or control over-eating and related disorders such as obesity.

Further, WO 2006/035446 teaches the stimulation of satiety by administration of an active agent specifically to a region where that active agent is preferentially active, for example, the duodenum, the antral sphincter and the gastrointestinal wall where chemoreceptors sensitive to the active agent are found. Suggested active agents include usually orally administered anti-obesity drugs such as lipase inhibitors, CCK, CCK analogs, GLP-1, PYY, beta-3-adrenergic agonists such as CL 316243 (White CL., et al., 2004, Physiol. Behav. 82(2-3): 489-96), antagonists to the cannabinoid receptor (Lichtman A H and Cravatt B F, 2005, J. Clin. Invest. 115: 1130-3) and fat derived weight maintaining drugs such as leptin.

WO 2006/035446 teaches a variety of methods for stimulating a region responsive to a gastrointestinal satiety, including administration of an active agent by irrigation, injection or dispersion through a dispersion tube. Although novel and effective, the teachings of WO 2006/035446 do not specifically teach an optimum method of stimulating the feeling of satiety.

It would be highly advantageous to have an efficient method and a device for efficiently preventing and/or treating obesity incorporating at least some of the teachings of WO 2006/035446.

More generally, it would be highly advantageous to have a method and a device for administering active agents such as active pharmaceutical ingredients that have advantages over the methods known in the art.

### SUMMARY OF THE INVENTION

The present invention successfully addresses at least some of the shortcomings of the prior art by providing a method and a device for administering sprayable pharmaceutical compositions including active agents such as active pharmaceutical ingredients (APIs) as a spray at a location in the gastrointestinal tract, the location generally selected as being preferred, ideal or in some way advantageous for administration of the active agent. In embodiments the active ingredient is administered at a selected moment, the moment selected as being preferred, ideal or advantageous in some way for administration of the active agent. In embodiments, administration is of active ingredients that stimulate a perception of satiety upon detection of a physiological change associated with food ingestion or hunger.

The present invention is based, at least in part, on the discovery that spray administration of a sprayable pharmaceutical composition (in some embodiments a fluid composition, in some embodiments a liquid composition, especially a non-viscous liquid composition) containing an active agent (e.g., a peptide such as a peptide hormone, analogue or derivative, especially a peptide having no more than 40 amino residues, no more than 30 amino acid residues and even no more than 20 amino acid residues) at a specific location of the gastrointestinal tract, especially at selected moments, is an unexpectedly effective manner of administering the active agent.

Thus, according to an aspect of some embodiments of the present invention there is provided for a method of administering an active agent, comprising: a) deploying a sprayer of a device for administering a sprayable pharmaceutical composition in a specific location of a gastrointestinal tract of a subject (in embodiments a human, in embodiments a non-human animal) suffering from a condition, preferably so that the sprayer is substantially fixed in place; b) providing a sprayable pharmaceutical composition comprising an active agent and a pharmaceutically acceptable carrier, the active agent effective in treating the condition; and c) when necessary, spraying a dose of the sprayable composition through the sprayer against a portion of a luminal wall of the gastrointestinal tract of the subject thereby administering the active agent so as to treat the condition.

In some embodiments, the method of the present invention is a topical method of administration, the active agent treating the condition, for example by interacting with chemoreceptors apparent on or in the gastrointestinal tract luminal wall.

In some embodiments, the method of the present invention is a systemic method of administration, the active agent treating the condition, for example, by being absorbed by the gastrointestinal tract so as to treat the condition.

In some embodiments, deploying the sprayer is such that the dose of spray is directed towards the luminal wall of the gastrointestinal tract.

In some embodiments, deploying the sprayer comprises anchoring the sprayer at the specific location, preferably substantially tensionless anchoring.

In some embodiments, the active agent is a peptide, such as a peptide hormone, an analogue thereof or a derivative thereof. In some embodiments the active agent is a peptide having no more than 40 amino residues, no more than 30 amino acid residues and even no more than 20 amino acid residues.

In some embodiments, the specific location of the gastrointestinal tract is selected from the group consisting of esophagus, stomach, antrum, antral sphincter, fundus, pylorus, small intestine, duodenum, jejunum, ileum, large intestine, caecum, vermiform appendix, colon, ascending colon, transverse colon, descending colon, sigmoid flexure and rectum. For example, in some embodiments, a peptide hormone (or an analogue thereof or a derivative thereof) is sprayed at the luminal wall of a duodenum (in embodiments, the superior portion of the duodenum) in order to interact with chemoreceptors apparent thereupon.

In some embodiments, the method further comprises functionally associating a reservoir holding the sprayable composition with the sprayer. In some embodiments, functionally associating the reservoir with the sprayer comprises deploying the reservoir in the gastrointestinal tract, for example, in the stomach. In some embodiments, functionally associating the reservoir with the sprayer comprises implanting the reservoir in the body, for example subcutaneously or in the abdomen.

Typical conditions from which the subject suffers for which the method of the present invention is useful include, but are not limited to obesity, bulimia, eating disorders, overeating, diabetes-related obesity, metabolic syndrome, inflammatory bowel disease, infections such as of helicobacter pylori, cardiovascular pathologies such as angina or arrhythmia, asthma and allergies.

In some embodiments, the necessity of administering the dose of the active agent is periodic, that is, doses are administered periodically according to a schedule, for example when the method of the present invention is used to provide maintenance doses of an active agent for treating a chronic condition.

In some embodiments, the necessity of administering the dose of the active agent is determined by detection of an event of significance for administration of the active agent and administration is initiated a specified time subsequent to detection of the event.

In some embodiments the detection of the event is non-automatic, for example is performed by a care-giver, medical professional or the subject self.

In some embodiments, the detection of the event is automatically performed with the help of an event detector functionally associated with the sprayer.

In some embodiments, the event is a physiological change, such as gastrointestinal activity indicative of an event such as food ingestion and hunger. In such embodiments, a typical sprayable composition comprises, as an active agent, a gastrointestinal satiety agent or anti-food absorption drug such as a lipase inhibitor

In some embodiments, the event comprises angina or arrhythmia, for example in a subject suffering from a cardiovascular pathology. In such an embodiment a typical active agent is an anti-anginal or anti-arrhythmic such as adenosine or nitrates (e.g., isosorbide dinitrate, isosorbide mononitrate or nitroglycerin) administered in the ileum.

In some embodiments, the event comprises an asthmatic attack, for example in a subject suffering from asthma. In such an embodiment a typical active agent is an anti-asthma agent such as a systemic bronchial activator (e.g., salbutamol) administered in the ileum.

In some embodiments, the event comprises an allergic reaction, especially a systemic allergic reaction, for example in a subject suffering from allergic reaction as a result of a bee sting or the like. In such an embodiment a typical active agent is an anti allergy agent (e.g., epinephrine) administered in the ileum.

In some embodiments, functionally associating a reservoir with the sprayer comprises deploying a reservoir in the gastrointestinal tract, especially in the stomach, and charging the reservoir with the sprayable composition. In some embodiments, the reservoir is periodically recharged with a sprayable composition while deployed in the gastrointestinal tract.

In some embodiments, functionally associating a reservoir with the sprayer comprises implanting a reservoir in the body of the subject, especially subcutaneously, and charging the reservoir with the sprayable composition. In some embodiments, the reservoir is periodically recharged with a sprayable composition while implanted.

In some embodiments, a method of administering an active agent of the present invention comprises: a) deploying a sprayer of a device of in a specific location of a gastrointestinal tract (e.g., the duodenum such as the superior portion of the duodenum) of a subject suffering from a condition; b) providing a sprayable pharmaceutical composition comprising a peptide active agent and a pharmaceutically acceptable carrier, the active agent effective in treating the condition; and c) when necessary, dispensing a dose of the sprayable composition through the sprayer so that the active agent interacts with chemoreceptors apparent on the luminal wall of the gastrointestinal tract of the subject thereby administering the active agent so as to treat the condition wherein the active agent is a peptide hormone, an analogue thereof or a derivative thereof. In some embodiments, the condition from which the subject suffers is a condition selected from the group comprising obesity, bulimia, eating disorders, overeating, diabetes-related obesity and metabolic syndrome. In some embodiments, the active agent is a gastrointestinal satiety agent. In some embodiments, the active agent is a CCK analogue or CCK derivative, or a CCK receptor agonist such as CCK-8. In some embodiments, the necessity of administering the dose of the active agent is determined by detection of an event of significance for administration of the active agent. In some embodiments, the event is a physiological change such as gastrointestinal tract activity, food ingestion or hunger. In some embodiments the peptide active agent has no more than 40 amino residues, no more than 30 amino acid residues and even no more than 20 amino acid residues.

An aspect of some embodiments of the present invention is of a device, for example suitable for implementing some embodiments of the method of the present invention.

Thus, according to an aspect of some embodiments of the present invention there is also provided a device for administering a sprayable pharmaceutical composition including an active agent to a luminal wall of the gastrointestinal tract of a subject, comprising: a) a sprayer configured for deployment (preferably substantially fixed deployment) in a gastrointestinal tract; b) a pressure generator configured to dispense a dose of a sprayable composition out through the sprayer as a spray upon actuation; and c) an actuator for actuating the pressure generator upon being triggered.

According to an aspect of some embodiments of the present invention there is also provided a device useful for administering a sprayable composition to a luminal wall of the duodenum of a subject, comprising: a) a sprayer configured for deployment in a duodenum; b) a feeder tube including a proximal end functionally associated with the sprayer and a distal end, the feeder tube configured to define a conduit for a sprayable composition from the distal end of the feeder tube to the sprayer; and c) an anchor functionally associated with the distal end of the feeder tube, wherein the anchor and the feeder tube are configured so that when properly deployed in the body of a subject, the feeder tube passes through the pyloric sphincter of the subject to maintain the sprayer in the duodenum of a subject.

According to some embodiments, the device further comprises an increased-diameter feature near the distal of the feeder tube, configured to assist in preventing the sprayer from passing through the pyloric sphincter into a stomach of a subject when properly deployed. According to some embodiments, the increased-diameter feature comprises the sprayer. According to some embodiments, the increased-diameter feature is expandable, easing deployment in a small-diameter conformation and preventing the passage back through the pyloric sphincter in an increased diameter conformation.

According to some embodiments, the feeder tube and the anchoring component are configured so that when properly deployed in the body of a subject, the feeder tube passes through the pyloric sphincter of the subject to maintain the sprayer in the superior portion of the duodenum of a subject.

According to some embodiments, the device further comprises a pressure generator functionally associated with the feeder tube which is configured to force sprayable composition out through the sprayer as a spray upon actuation of the pressure generator. Depending on the embodiments, the pressure generator is configured for deployment inside the gastrointestinal tract, for deployment in the abdominal cavity, for subcutaneous deployment, for deployment outside the body of the subject, or for deployment elsewhere.

According to some embodiments, the device further comprises a sprayable composition reservoir functionally associated with the pressure generator, wherein the sprayable composition forced out through the sprayer by the pressure generator is sprayable composition held in the reservoir. Depending on the embodiment, the pressure generator is configured for deployment inside the gastrointestinal tract, for deployment in the abdominal cavity, for subcutaneous deployment, for deployment outside the body of the subject, or for deployment elsewhere.

According to some embodiments, where the pressure generator and/or the sprayable composition reservoir are not configured to be deployed in the gastrointestinal tract (e.g., when configured for deployment in the abdominal cavity, subcutaneously or outside of the body of a subject), the feeder tube is configured to pass through an opening in a wall of the gastrointestinal tract.

In some embodiments, the sprayer comprises at least one orifice. In some embodiments, at least one orifice is a nozzle. In some embodiments, at least one orifice is a slit. In some embodiments, the at least one the orifice is configured to direct a spray towards a luminal wall of a gastrointestinal tract in which the sprayer is deployed (e.g., the sprayer has an axis and the orifice is configured to direct a spray away from an axis of the sprayer). In some embodiments, the sprayer comprises at least two orifices configured to direct a spray towards a luminal wall of a gastrointestinal tract in which the sprayer is deployed (e.g., the sprayer has an axis and the orifices are configured to direct a spray away from an axis of the sprayer) each in a different direction away from an axis of the sprayer.

In some embodiments, at least one (preferably all) of the orifice is configured to function as a valve allowing the sprayable composition to be forced out through the sprayer upon actuation of the pressure generator but substantially preventing passage of fluids through the orifice when the pressure generator is not actuated.

In some embodiments, the device is configured so that, when deployed, the sprayer is suspended in the lumen of the gastrointestinal tract.

In some embodiments, the device is configured so that, when deployed, a portion of the sprayer is near to or contacting a part of the luminal wall of the gastrointestinal tract. In some embodiments, the sprayer is configured (for example by positioning and shape of orifices) to spray towards a luminal wall in proximity of the part of the luminal wall that the portion of the sprayer contacts or is near to.

In some embodiments, a sprayer of the device is configured to spray a region of no less than 1 cm long of a luminal wall in which the sprayer is deployed, for example by including a plurality of orifices along a length of the sprayer or by providing orifices having a relatively large arc with respect to the length of the gastrointestinal tract. In some embodiments the length of the region sprayed is of no less than 2 cm, of no less than 3 cm and even of no less than 4 cm.

In some embodiments, the device is configured so that the sprayer is deployable at a specific location in a gastrointestinal tract, for example in the esophagus, stomach, antrum, antral sphincter, fundus, pylorus, small intestine, duodenum, jejunum, ileum, large intestine, caecum, vermiform appendix, colon, ascending colon, transverse colon, descending colon, sigmoid flexure or rectum. In some embodiments, the device is provided with a marker (e.g., a feature or component) that is observable and allows determination of the location of the sprayer in the gastrointestinal tract. In some embodiments, a marker is radio-opaque. In some embodiments, a marker is ultrasound opaque.

In some embodiments, the device further comprises e) an anchor, configured to anchor the sprayer at a specific location in the gastrointestinal tract.

In some embodiments, the sprayer is in fluid communication with the pressure generator through a feeder tube, allowing transport of a fluid from the pressure generator to the sprayer. Generally the size of the bore of the feeder tube is determined by the length of the feeder tube from the pressure generator, the properties of the sprayer, the properties of the pressure generator and the viscosity of the composition which the device is configured to spray. In some embodiments, the outer diameter of the feeder tube is no more than about 4 mm, no more than about 3 mm and even no more than 2 mm. In some embodiments, a feeder tube is configured, at least in part, to assist in anchoring and/or positioning the sprayer in a desired location in the gastrointestinal tract. For example, in some embodiments, at least portion of a feeder tube is coiled so as, when deployed in the gastrointestinal tract, the coiled portion of the feeder tube presses against the luminal wall of the gastrointestinal tract to position or anchor the sprayer.

In some embodiments, the pressure generator is selected from the group consisting of spring powered pressure generators, motor powered pressure generators and gas pressure powered pressure generators. In some embodiments, the device further comprises a power supply unit for providing power for operation of the pressure generator. Typical power supply units include power storage units (especially rechargeable power storage units such as rechargeable batteries) and power generation units.

In some embodiments, the device further comprises d) a composition reservoir functionally associated with the pressure generator and the pressure generator is configured to force a sprayable composition held in the reservoir from the reservoir out through the sprayer as a spray upon the actuation. In some embodiments, the reservoir is configured for deployment inside the body of a subject and in some embodiments is even configured to be recharged when deployed inside the body. In some embodiments, the reservoir is configured for deployment in a gastrointestinal tract, preferably in a stomach. In some embodiments, the reservoir is configured for recharging with composition when deployed in a gastrointestinal tract. In some embodiments, the reservoir is configured for implantation in the body of a subject, preferably subcutaneously. In some embodiments, the reservoir is configured for recharging with composition when implanted in the body. In some embodiments, the reservoir is configured for deployment outside of the body of a subject.

In some embodiments, a device of the present invention further comprises a pharmaceutical composition including a peptide active agent (especially having no more than 40 amino residues, no more than 30 amino acid residues and even no more than 20 amino acid residues) and a pharmaceutically acceptable carrier held in a reservoir. In some embodiments the active agent is a peptide hormone, an analogue thereof or a derivative thereof. In some embodiments, the active agent is configured to interact with chemoreceptors apparent on luminal walls of a portion of a gastrointestinal tract in which the sprayer is to be deployed.

In some embodiments, the actuator is configured to actuate the pressure generator so as to dispense a specified dose of sprayable composition. In some embodiments the dose is a fixed dose. In some embodiments, the device further comprises a dosage adjusting mechanism functionally associated with the actuator and/or with the pressure generator.

In some embodiments, the device further comprises f) an event detector functionally associated with the actuator, configured so that as a result of detection of an event of significance for administration of an active agent, the event detector triggers the actuator.

In some embodiments, the event is a physiological change. Typical physiological changes for example gastrointestinal tract activity indicative of an event such as food ingestion, hunger, anginal attack, arrhythmia, an asthma attack or an allergic reaction especially a systemic allergic reaction.

In some embodiments, the event detector comprises an electrode configured for deployment in the body.

In some embodiments, the device comprises a timer functionally associated with the actuator and with the event detector and the timer is configured to trigger the actuator a specified period of time subsequent to the detection of the event by the event detector.

In some embodiments, the device comprises a timer functionally associated with the actuator and, for example, the timer is configured to periodically trigger the actuator.

In some embodiments, a timer is configured to trigger the actuator for a specified period of time. In some embodiments, a timer is adjustable, that is, the period of time over which a sprayable composition is administered or the time delay after detection of an event which the sprayable composition is administered is changeable.

In some embodiments, the device for administering a sprayable pharmaceutical composition including an active agent to a luminal wall of the gastrointestinal tract of a subject, comprises: a) a sprayer configured for substantially fixed deployment in a gastrointestinal tract, the sprayer comprising at least one orifice configured to direct a spray towards a portion of the luminal wall of a gastrointestinal tract (preferably of the duodenum) in which the sprayer is deployed; b) a pressure generator configured to dispense a dose of sprayable composition out through the sprayer as a spray upon actuation; c) an actuator for actuating the pressure generator upon being triggered; d) a composition reservoir functionally associated with the pressure generator so that the pressure generator is configured to force sprayable composition from the reservoir out through the sprayer as a spray upon actuation; and further comprising a sprayable pharmaceutical composition including a peptide active agent (especially having no more than 40 amino residues, no more than 30 amino acid residues and even no more than 20 amino acid residues) and a pharmaceutically acceptable carrier held in the reservoir. In some embodiments, the device further comprises d) an event detector functionally associated with the actuator, configured so that as a result of detection of an event of significance for administration of an active agent, the event detector triggers the actuator. In some embodiments, the event is a physiological change indicative of food ingestion and/or hunger. In some embodiments, the active agent is configured to interact with chemoreceptors apparent on luminal walls of the portion of the gastrointestinal tract. In some embodiments, the active agent is a peptide hormone, an analogue thereof or a derivative thereof. In some embodiments, the active agent is a gastrointestinal satiety agent, such as a CCK analogue or CCK derivative, such as CCK-8.

It has further been found that the teachings of the present invention allow gastrointestinal administration of composition including peptide active ingredients such as peptide hormones for treating medical conditions without the peptides being substantially digested in the gastrointestinal tract before having a desired physiological effect.

Thus, according to an aspect of some embodiments of the present invention there is also provided a method of treatment, comprising: a) providing a sprayable pharmaceutical composition which comprises a peptide active agent and a pharmaceutically acceptable carrier; and b) administering the composition to a subject in need thereof by spraying the composition in a part of the gastrointestinal tract of the subject so that the active agent interacts with the luminal wall of the gastrointestinal tract thereby causing a beneficial effect. In some embodiments, the luminal wall is the luminal wall of the duodenum of the subject. In some embodiments, the peptide is a peptide hormone, an analogue thereof or a derivative thereof. In some embodiments, at least some of the beneficial effect results from the interaction of the active agent with chemoreceptors apparent on the luminal wall. In some embodiments, the peptide has no more than 40 amino residues, no more than 30 amino acid residues and even no more than 20 amino acid residues. In some embodiments, the active agent is a peptide hormone, an analogue thereof or a derivative thereof. In some embodiments, the active agent is a gastrointestinal satiety agent. In some embodiments, the active agent is a CCK analogue or CCK derivative such as CCK-8.

Thus, according to an aspect of some embodiments of the present invention there is also provided a method of treatment, comprising: a) providing a sprayable pharmaceutical composition which comprises a satiety factor or an analogue thereof or a derivative thereof as an active agent and a pharmaceutically acceptable carrier; and b) administering the composition to a subject in need thereof by spraying the composition in a portion of the gastrointestinal tract of the subject so that the active agent interacts with chemoreceptors apparent on the luminal wall of the portion of the gastrointestinal tract, thereby causing a beneficial effect. In some embodiments the luminal wall is the luminal wall of the duodenum of the subject. In some embodiments the subject suffers from a disorder selected from the group consisting of obesity, bulimia, eating disorders, overeating, diabetes-related obesity and metabolic syndrome. In some embodiments, the beneficial effect is a reduction of calories consumed and/or a reduction of the amount food consumed. In some embodiments, the satiety factor is CCK or an analogue thereof or a derivative thereof such as CCK-8.

Thus according to an aspect of some embodiments of the present invention there is also provided for the use of a peptide (especially a peptide having no more than 40 amino residues, no more than 30 amino acid residues and even no more than 20 amino acid residues) as an active agent for the manufacture of a sprayable pharmaceutical composition (that is to say a sprayable medicament), for use in the treatment of a subject by gastrointestinal administration (especially duodenal administration). In some embodiments, the peptide is a peptide hormone, an analogue thereof or a derivative thereof.

Thus, according to an aspect of some embodiments of the present invention there is also provided for the use of a peptide (especially having no more than 40 amino residues, no more than 30 amino acid residues and even no more than 20 amino acid residues) as an active agent for the manufacture of a sprayable pharmaceutical composition (that is to say a medicament) for use in the treatment of a subject by gastrointestinal administration (especially duodenal administration), wherein the peptide is configured to interact with chemoreceptors apparent on the surface of the gastrointestinal tract. In some embodiments, the treatment comprises reduction of calorie intake by the subject and or reduction of the amount of food consumed by a subject. In some embodiments, the subject suffers from a disorder selected from the group consisting of obesity, bulimia, eating disorders, overeating, diabetes-related obesity and metabolic syndrome. In some embodiments, the peptide is a peptide hormone, an analogue thereof or a derivative thereof. In some embodiments, the peptide is a gastrointestinal satiety agent, such as a CCK analogue or CCK derivative such as CCK-8.

In some embodiments of the uses above, the sprayable composition is configured for treating a condition selected from the group consisting of obesity, bulimia, eating disorders, overeating, diabetes-related obesity, metabolic syndrome, inflammatory bowel disease, infections such as of helicobacter pylori, cardiovascular pathologies such as angina or arrhythmia, asthma and allergies.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Herein, the term “active agent” is meant to include chemical, biological or pharmaceutical materials including any natural or synthetic chemical or biological substance that influences a cell, an organ or organism to which the material is administered. Typical active agents include but are not limited to active pharmaceutical ingredients, antibodies, antigens, biological materials, chemical materials, chemotherapeutic agents, diagnostic agents, DNA, drugs, dyes, enzymes, foodstuffs, hormones, immunogenes, ligands, liposomes, markers, nanoparticles, nucleic acids, nutrients, physiological media, proteins, radio-labeled markers, RNA, selective toxins, therapeutic monoclonal antibodies, toxins and vaccines and especially peptides and peptide hormones.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of some embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A, 1B, 1C and 1D are schematic depiction of a first embodiment of a device of the present invention;

FIG. 2 is a schematic depiction of the device of FIG. 1 deployed in a gastrointestinal tract;

FIG. 3 is a schematic depiction of an embodiment of a second embodiment of a device of the present invention deployed in a gastrointestinal tract;

FIGS. 4A, 4B and 4C are schematic depiction of a second embodiment of a device of the present invention;

FIGS. 5A and 5B are schematic depictions of head-on cross sections of embodiments of sprayers suitable for implementing the teachings of the present invention;

FIGS. 6A, 6B, 6C and 6D are schematic depictions of side cross sections of embodiments of sprayers suitable for implementing the teachings of the present invention;

FIG. 7 is a schematic depiction of a device used to test the teachings of the present invention;

FIG. 8 is a graph displaying the effect of administration of CCK-8 in accordance with the teachings of the present invention on gallbladder contraction;

FIG. 9 is a graph displaying the effect of administration of CCK-8 in accordance with the teachings of the present invention on caloric consumption;

FIG. 10 is a graph displaying the effect of administration of CCK-8 in accordance with the teachings of the present invention on food consumption;

FIG. 11 is a graph displaying the effect of administration of CCK-8 in accordance with the teachings of the present invention on food taken; and

FIGS. 12A-12E depict an embodiment of a device of the present invention where a feeder tube passes from outside the body of a subject, into the stomach cavity, through the pyloric sphincter to a sprayer deployed in the duodenum.

### DESCRIPTION OF EMBODIMENTS

Some embodiments of the present invention are of methods and devices for the administration of an active agent for treating a condition. Specifically, some embodiments of the methods and the devices of the present invention relate to administration of a sprayable pharmaceutical composition including an active agent by deploying a sprayer at a specific location in the gastrointestinal tract and, when needed, spraying the composition through the sprayer at a luminal wall of the gastrointestinal tract. Some embodiments of the present invention also relate to the use of peptides, such as peptide hormones, such as CCK, CCK analogues and derivatives thereof in the preparation of pharmaceutical compositions (medicaments).

According to an embodiment of a method of the present invention for administering an active agent, a sprayer is deployed in a specific location of a gastrointestinal tract of a subject suffering from a condition, preferably so that the sprayer is substantially fixed in place and a sprayable pharmaceutical composition comprising an active agent effective in treating the condition is provided. The specific location is selected as being a location that is in preferred, ideal or in some way advantageous for administration of the active agent, for example the location is where the administered active agent is ideally absorbed, where chemoreceptors sensitive to the active agent are found or where administration exhibits the fewest side effects.

Although not wishing to be held to any one theory, it is believed that administering a composition by spraying the composition onto a surface where receptors are found (for example, receptors found on the duodenal lumen) increases the availability of an active agent to the receptors, increases absorption and more evenly disperses the composition.

When necessary, a dose of a sprayable pharmaceutical composition (in some embodiments a fluid composition, in some embodiments a liquid composition, especially a non-viscous liquid composition) is sprayed through the sprayer against a portion of a luminal wall of the gastrointestinal tract of the subject thereby administering the active agent so as to treat the condition.

By treating the condition are included, but not limited to, curing the condition, treating the condition, preventing the condition, treating symptoms of the condition, curing symptoms of the condition, ameliorating symptoms of the condition, treating effects of the condition, ameliorating effects of the condition, and preventing results of the condition.

By dose is meant a pharmaceutically effective amount of sprayable pharmaceutical composition that includes the active agent. By “pharmaceutically effective amount” is meant describes an amount of the composition that is sufficient to lead to a desired effect in the condition being treated, but low enough to avoid significant side effects, within the scope of sound judgment of a health care professional such as a medical doctor. The effective amount of the composition may vary with the particular area being treated, the age and physical condition of the subject being treated, the severity of the condition, the duration of the treatment, the nature of concurrent therapy, the specific active agent employed, the particular carrier utilized, and like factors within the knowledge and expertise of one skilled in the art.

As used herein a “pharmaceutical composition” refers to a preparation of one or more of the active agents with other components such as physiologically suitable carriers and excipients. The purpose of a pharmaceutical composition is to facilitate administration of a compound to a subject. Formulation of a sprayable pharmaceutical composition and determination of a suitable dose is within the ability of one of average skill in the art using techniques with which one of average skill is familiar which are discussed in numerous reference works such as Remington's Pharmaceutical Science 15th Edition and generally includes mixing an amount of the active agent with another material or materials, such as excipients and carriers.

Hereinafter, the phrases “physiologically acceptable carrier” and “pharmaceutically acceptable carrier” which may be interchangeably used refer to a carrier or a diluent that does not cause significant irritation to a subject and does not abrogate the biological activity and properties of the administered active agent. An adjuvant is included under these phrases.

Herein the term “excipient” refers to an inert substance added to a pharmaceutical composition to further facilitate administration of an active agent. Examples, without limitation, of excipients include calcium carbonate, calcium phosphate, various sugars and types of starch, cellulose derivatives, gelatin, vegetable oils and polyethylene glycols.

Pharmaceutical compositions used in implementing the teachings of the present invention may be formulated in conventional manner using one or more physiologically acceptable carriers comprising excipients and auxiliaries, which facilitate processing of the active entities into pharmaceutical compositions. Suitable techniques are described in “Remington's Pharmaceutical Sciences,” Mack Publishing Co., Easton, Pa., latest edition, which is incorporated herein by reference. For example, pharmaceutical compositions of the present invention may be manufactured by one or more processes that are well known in the art, e.g., mixing, blending, homogenizing, dissolving, granulating, emulsifying, encapsulating, entrapping and lyophilizing processes. For example, sprayable compositions of the present invention may include aqueous solutions, in some embodiments in physiologically compatible buffers such as Hank's solution, Ringer's solution, or physiological salt buffer. For transmucosal administration, penetrants appropriate to the barrier to be permeated are used in the formulation. Such penetrants are generally known in the art.

Pharmaceutical compositions suitable for use in the context of the present invention generally include compositions comprising active agents in an amount effective to achieve the intended purpose, for example in some embodiments a therapeutically effective amount means an amount of active agent (e.g., a satiety drug or an anti-food absorption drug) effective in reducing appetite. Determination of a therapeutically effective amount is well within the capability of those skilled in the art, especially in light of the detailed disclosure provided herein. When implementing the teachings of the present invention, a therapeutically effective amount or dose can be estimated initially from animal models such as monkey or pigs. For example, a dose can be formulated in animal models to achieve a desired concentration or titer. Such information can be used to more accurately determine useful doses in humans.

For example, a dose of CCK-8 can be in the range of 0.04 to 0.4 micrograms per kg body weight. Thus, for an individual who weighs 125 kg, such a dose can be for example of 10 micrograms CCK-8.

Exemplary conditions from which the subject suffers for which some embodiments of the method of the present invention may be useful include, but are not limited to obesity, bulimia, eating disorders, overeating, diabetes-related obesity, metabolic syndrome, inflammatory bowel disease, helicobacter pylori infection, cardiovascular pathologies, asthma and allergy.

In some embodiments, the method of the present invention is a topical method of administration, that is to say the active agent acts for example by interacting with chemoreceptors apparent on or in the gastrointestinal tract luminal wall or the active agent acts by treating a pathology apparent on the gastrointestinal tract luminal wall In some embodiments, the method of the present invention is a systemic method of administration, that is to say the active agent is absorbed by the organism through the gastrointestinal tract in order to act.

In some embodiments, the necessity of administration of the active agent is part of a regular administration protocol, for example a prophylactic or maintenance dose, for example for the treatment of a chronic condition. In some embodiments, a dose of the active agent is administered periodically, that is, doses are administered according to a substantially fixed schedule. Depending on the severity and responsiveness of the condition to be treated, a given treatment regime may by chronic, last from several days, several weeks or until a desired effect is achieved. It will be appreciated that an efficient dose can be adjusted to the treated individual based. That said, the treatment regime (e.g., dosage, frequency) of a composition be administered will, of course, be dependent on the subject being treated, the severity of the subject and the judgment of the responsible health-care professional.

In some embodiments, administration of the sprayable composition is initiated and performed manually (e.g., by a caregiver, a health-care professional or the subject self) while in some embodiments administration is initiated and performed automatically.

In some embodiments, administration of the active agent is event-driven, that is a dose of active agent is administered subsequent to the detection of some event of significance for administration of the active agent, for example physiological changes. In some embodiments related to eating disorders typical significant events include detection of muscle activity (e.g., of the stomach), pressure (e.g., caused by stomach contractions or resulting from food entering the stomach or esophagus), change of chemical composition such as pH (e.g., from the release of enzymes, acids or other gastric juices), body temperature and electrical currents in the vagus nerves or pancreas when a person is hungry or consuming food. In some embodiments related to the treatment of a subject suffering from a cardiovascular condition typical significant events include detection of an anginal attack or arrhythmia. In some embodiments related to the treatment of a subject suffering from asthama typical significant events include detection of an asthma attack. In some embodiments related to the treatment of a subject suffering from an allergy typical significant events include detection of an allergic reaction, especially a systemic allergic reaction.

In some embodiments, detection of an event is manual, that is to say, includes at least one step performed by a person, such as a caregiver, a health-care professional or the subject self. For example, a subject suffering from asthma identifies an asthmatic attack and initiates active agent administration. In some embodiments, detection of an event is automatically performed, for example with the help of an event detector that is functionally associated with the sprayer.

In some embodiments, the method of the present invention further comprises functionally associating a reservoir holding the sprayable composition with the sprayer, allowing the method to be performed unobtrusively, autonomously, automatically and with minimal inconvenience for the subject. In some embodiments, functionally associating the reservoir with the sprayer comprises deploying the reservoir in the gastrointestinal tract, for example, in the stomach. In some embodiments, functionally associating the reservoir with the sprayer comprises implanting the reservoir in the body of the subject, for example, subcutaneously.

The passage of an active agent orally administered in accordance with the prior art through the gastrointestinal tract is a substantially continuous process where the administered active agent is diluted in the gastrointestinal fluids. Thus, the concentration of the active agent in the gastrointestinal fluid is indeterminate and varies as a result of many factors including the volume of the fluid as well as the composition of the fluid which may include fibers and other molecules which may absorb, adsorb or otherwise influence the active agent. Further, the pH of the gastrointestinal fluid changes the ratio of ionized/un-ionized active agent. In addition, the varying rate of peristalsis that changes the flow rate through the gastrointestinal tract combined with the variable dilution makes the effective concentration of an active agent at the lumen wall indeterminate and difficult to control. The result is that an administered dose of an active agent may often be too low (leading to insufficient efficacy) or too high (leading to any one of numerous negative effects) while a significant proportion of an active agent is carried through the gastrointestinal tract past sites such as chemoreceptors where the active agent is effective.

The teachings of some embodiments of the present invention are based, at least in part, on the discovery that spray-administration of a sprayable composition containing an active agent at a specific location of the gastrointestinal tract, especially at carefully selected moments, is an unexpectedly effective manner of administering the active agent, the spraying overcoming many of the challenges of gastrointestinal tract delivery of active agents.

Although not wishing to be held to any one theory, it is believed that administration in accordance with the teachings of the present invention brings an administered active agent in contact with a luminal wall of the gastrointestinal tract where the pharmaceutical or other beneficial activity of the active agent occurs at a significantly more controlled effective dosage in a relatively well defined chemical and physical environment as the spraying reduces mixing with the gastrointestinal liquids. Thus, the teachings of the present invention apparently provide for administering an active agent at a controlled concentration under controlled chemical conditions to a well-determined location in the gastrointestinal tract. As a result, some embodiments of the present invention allow administration of a lesser amount of active agent reducing potential side effect. Further, the chance of overdosing is reduced as in some embodiments, the time between administration and an actual effect is generally short.

An additional unexpected effect is the surprising efficacy with which peptides, such as peptide hormones, are administered. It is known that it is challenging to administer peptides as active agents through the gastrointestinal tract as these are quickly digested and rendered ineffective by the chemical conditions inside the gastrointestinal tract. Further, peptide absorption is generally affected by pH due to the ease with which peptides are ionized. The present invention therefore provides an unexpectedly convenient method for administering peptide active agents in pharmaceutical compositions and in medicaments. Further, in some embodiments the present invention allows for the use of active agents such as peptides in the preparation of pharmaceutical compositions (medicaments), especially pharmaceutical compositions (medicaments) that are substantially devoid of peptidase inhibitors and the administration of such pharmaceutical compositions in accordance with the teachings of the present invention.

The term “peptide” as used herein encompasses native peptides and their analogues (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells, especially peptides that are not longer than 40, not longer than 30 and even not longer than 20 amino acid residues long. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), *-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g., fatty acids and complex carbohydrates.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Peptides used in implementing the teachings of the present invention may be linear or cyclic.

Peptides used in implementing the teachings of the present invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. In general, such methods include the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of the present invention involves solid phase peptide synthesis.

Large scale peptide synthesis is described by Andersson in Biopolymers 2000; 55(3):227-50.

In some embodiments, the present invention provides for the effective administration of gastrointestinal satiety agents or derivatives thereof or analogs thereof that mimic the physiological action of the gastrointestinal satiety agent. Such active agents include naturally occurring or synthetic hormones, peptides, neurotransmitters or mimetic thereof, that are capable of directly stimulating the region responsive to the satiety agent (e.g., the duodenum). Specific active agents for which administration using the teachings of the present invention is exceptionally useful include but are not limited to peptide hormones, CCK (GenBank Accession No. NP_{—}000720), Bombesin, Gastrin releasing peptide (GRP), glucagon, Enterostatin, Ghrelin, GLP-1 (glucagon-like peptide) (Bojanowska E., 2005, Med. Sci. Monit. 11:RA271-8; BYETTA™ (exenatide)), PYY (le Roux CW., et al., 2005, Endocrinology. 2005 Sep. 15; GenBank Accession No. NP_{—}004151), Oxyntomodulin (OXY, OXM; GenBank Accession No. P01275; Stanley S., et al., 2004, Am. J. Physiol. Gastrointest. Liver Physiol. 286(5): G693), Apo IV (naturally occurring apoprotein Qin X, Tso P 2005, Curr Drug Targets. 6(2):145-51), GII81771X (GSK), anti Ghrelin agents (Kobelt P., Gut. 2005 Jun. 30; SPIEGELMER NOX-B11), PP (Miskowiak J, et al., 1985, Regul. Pept. 12: 231-6) and derivatives and analogs thereof such as CCK-4 (Trp-Met-Asp-Phe), CCK-8 (Asp-Tyr(SO₃H)-Met-Gly-Trp-Met-Asp-Phe) analogues of CCK), CCK analogs ((Sincalide by Bracco Diagnostics or Squibb Diagnostics), GSK—GW7176, GW 5283, GW7854 and Pfizer PW170292)), CCK receptor agonists (e.g., 1,5-benzodiazepines, PD 170292, SR 146131) and/or activator molecules of the CCK-A receptor (JMV 180; Archer-Lahlou E, et al., 2005, J. Biol. Chem., Vol. 280: 10664-10674), and PYY analogs (e.g., PYY(1-36), PYY(3-36), PYY(9-36), PYY(14-36), PYY(22-36), and PYY(27-36)).

Some embodiments of the present invention actualize a central concept taught in the PCT patent application published as WO 2006/035446 of the Applicant by the unexpected discovery that stimulation of gastrointestinal chemoreceptors with an active agent such as an API (e.g., a peptide hormone) is exceptionally effective when the active agent is administered by spraying a composition including the active agent at the luminal wall of the gastrointestinal tract where the chemoreceptors are found. The spray provides for a quick administration of a layer of composition including an active agent over a large surface area of the luminal wall with relatively little composition wasted by dilution in the gastrointestinal fluid.

Some embodiments of the present invention have further significant advantages. As a dose is not necessarily prepackaged, but is rather determined by an amount of sprayable composition sprayed, in some embodiments a dose administered to a subject is personalized and can be adjusted for increased efficacy. As compositions are administered according to the teachings of the present invention directly in the gastrointestinal tract and do not pass the mouth and other sensitive tissue, compositions different than those known in the art, for example compositions that are bitter, oily or otherwise unpalatable may be administered. In some embodiments, compositions including low solubility active agents are administered by administering greater volumes of a composition or by administering compositions having a higher than usual organic solvent (e.g., ethanol, propylene glycol) content. In some embodiments, complex administration protocols may be formulated (e.g., multiple times daily or at exact intervals or including two or more separate active agents). Some embodiments of the present invention including automatic administration and especially also including automatic administration triggered by automatic event detection virtually guarantee absolute patient compliance. Some embodiments of the present invention are exceptionally suitable for chronic administration, by ensuring long-term patient compliance and by efficient use of a given amount of active agent to reduce side effects. In some embodiments of the present invention including automatic administration of a composition triggered by automatic event detection, treatment is exceptionally effective as an event may be detected before significant damage is done and a precise dose of active agent is administered without overdosing.

An aspect of the present invention is of a device suitable for administering a sprayable pharmaceutical composition including an active agent to a luminal wall of the gastrointestinal tract, for example, for implementing some embodiments of the method of the present invention. Generally, some embodiments of a device of the present invention comprise: a) a sprayer configured for deployment in a gastrointestinal tract; b) a pressure generator (e.g., a pump, such as a mechanical pump, an electrical pump, an pressurized gas-powered pump) configured to dispense a dose of sprayable composition out through the sprayer as a spray upon actuation; and c) an actuator for actuating the pressure generator upon being triggered.

Some embodiments of the sprayer of a device of the present invention include one or more orifices that are configured to direct a spray in one or more directions towards a luminal wall of a gastrointestinal tract in which the sprayer is deployed.

Some embodiments of a device of the present invention comprise an event detector to automatically detect an event of significance for administration of an active agent and, upon detection of such an event, to automatically trigger the actuator so as to administer a dose of the sprayable pharmaceutical composition.

Some embodiments of a device of the present invention comprise a composition reservoir functionally associated with the pressure generator and the pressure generator is configured to force sprayable composition from the reservoir out through the sprayer as a spray upon the actuation. In some embodiments, the reservoir is configured for deployment in a gastrointestinal tract, preferably in a stomach. This allows the device to be deployed in the gastrointestinal tract of a subject unobtrusively and with minimal inconvenience and to function automatically. In some embodiments, the reservoir is configured for implantation in the body of the subject, preferably subcutaneously. This allows the device to be deployed in the body of a subject unobtrusively and with minimal inconvenience and to function automatically.

The principles of the method and the device of the present may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other some embodiments or of being practiced or carried out in various ways. Also, it is to be understood that the phraseology and terminology employed herein is for the purpose of description and should not be regarded as limiting.

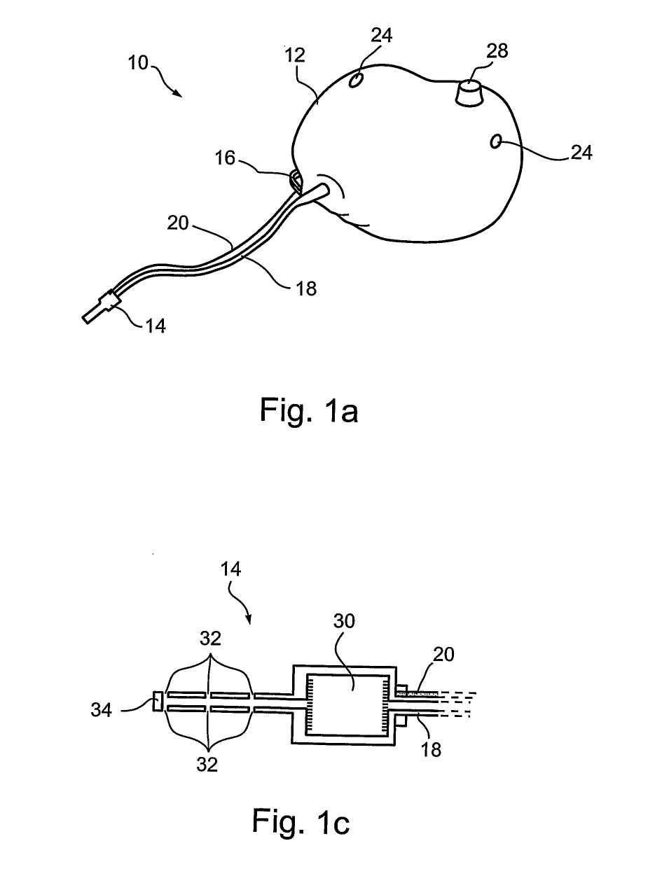

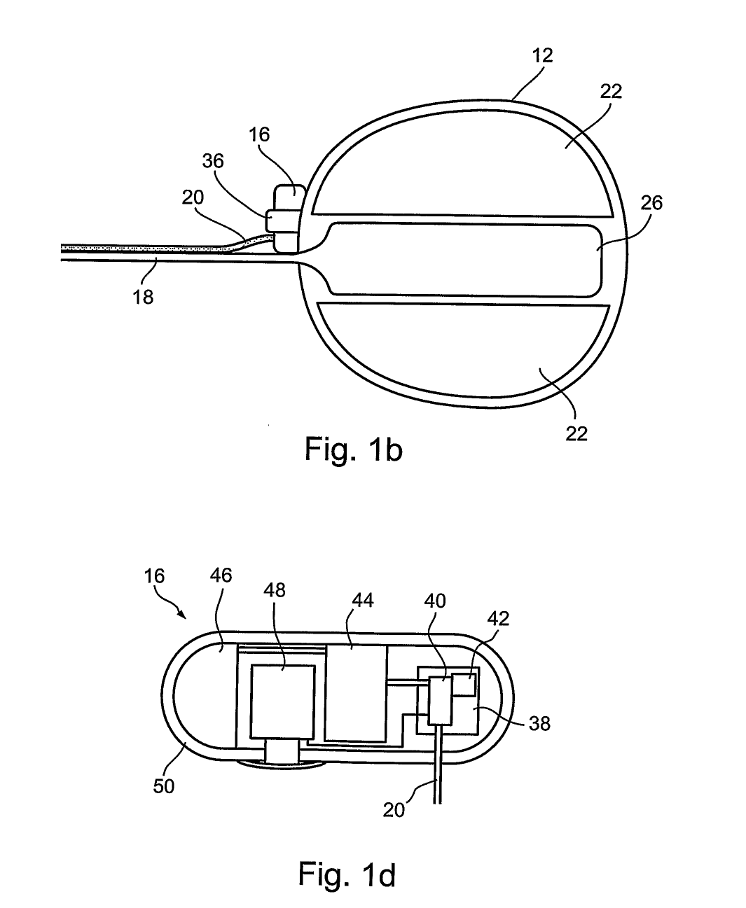

In FIGS. 1A, 1B, 1C and 1D are depicted an embodiment of the device of the present invention, device 10 configured for treating conditions relating to eating disorders for which administration of satiety agents may be beneficial. Device 10 comprises a number of components including reservoir 12, spray head 14, control unit 16, feeder tube 18 and power line 20.

Reservoir 12, depicted in cross section in FIG. 1B, is substantially a three-chambered elastic balloon configured for deployment in the stomach and is similar in construction and made of materials known in the art of intragastric balloons, e.g., BioEnterics® Intragastric Balloon System (Inamed Health, a division of Allergan, Santa Barbara, Calif., USA) or as discussed in the PCT patent application published as WO 2006/035446. Reservoir 12 comprises two space-filling chambers 22 each associated with a charging port 24 and a composition chamber 26 associated with a charging port 28 and in fluid communication with feeder tube 18. Chambers 22 and 26 are ordinarily in a collapsed state but stretch outwards when a fluid, such as a gas or liquid, is introduced into a chamber through a respective charging port 24 or 28. When entirely filled, space-filling chambers 22 occupy a volume of approximately 300 ml. When entirely filled, composition chamber 26 occupies a volume of approximately 100 ml.

Spray head 14, depicted in cross section in FIG. 1C, comprises a pressure generator 30 (an electrically powered pump, for example similar to pumps used in the field of insulin administration) that takes a sprayable composition from feeder tube 18 and forces the composition out as a spray through nozzles 32 (six nozzles 32 are depicted in FIG. 1C) of sprayer 34. As is seen in FIG. 1C, nozzles 32 are positioned and configured so that a spray exiting from nozzles 32 is directed away from the axis of sprayer 34 and thus sprayer 34 is configured to direct a spray towards the luminal wall of a gastrointestinal tract in which sprayer 34 is deployed, where nozzles 32 are arranged to spray in at least two different directions away from the axis.

Control unit 16 is depicted in FIGS. 1A and, 1B secured to reservoir 12 with an elastic band 36. In FIG. 1D components of control unit 16 are depicted: actuator 38 comprising controller 40 and timer 42, power storage unit 44, power storage unit charger 46, event detector 48 and casing 50.

Event detector 48 comprises a pressure sensor that is configured to detect contractions of a stomach in which reservoir 12 is deployed, contractions which are indicative of an event such as hunger or food ingestion. When such an event is detected, event detector 48 transmits the fact of detection to controller 40.

Power storage unit 44 is a rechargeable battery functionally associated with components of device 10 that require power such as actuator 38 and especially with pressure generator 30 of spray head 14 through actuator 38 and power line 20.

Power storage unit recharger 46 comprises a photovoltaic cell and other necessary components and is configured to convert near-infrared light impinging on the photovoltaic cell to electricity to recharge power storage unit 44.

Controller 40 comprises a populated circuit board with appropriate electronic components and is functionally associated with timer 42 to constitute actuator 38 and connects between power storage unit 44 and pressure generator 30 of spray head 14 through power line 20. Amongst other functions, controller 40 is configured so that upon receipt of a signal that an event is detected from event detector 48, controller 40 allows power to pass from power storage unit 44 to pressure generator 30 of spray head 14 for a specified time with reference to timer 42.

Casing 50 is substantially impervious to conditions in the stomach and comprises a two-part welded shell of a fluorocarbon polymer that is substantially transparent to light having near-infrared wavelengths that is converted by power storage unit recharger 46 to electricity.

Feeder tube 18 provides fluid communication between composition chamber 26 of reservoir 12 and spray head 14. Feeder tube 16 is substantially a hollow tube of a material resistant to the condition of the gastrointestinal tract (e.g., a fluorocarbon polymer such as polytetrafluoroethylene). The length of feeder tube 16 is such that, when reservoir 12 is deployed in a stomach, spray head 14 lays in and is thus deployed in the descending portion of the duodenum.

Power line 20 is substantially a cable that provides power from control unit 18 to pressure generator 30.

An embodiment of the method of the present invention applied for the treatment of an eating disorder (e.g., obesity, bulimia, eating disorders, overeating, diabetes-related obesity, metabolic syndrome) will be described with reference to device 10, as described above.

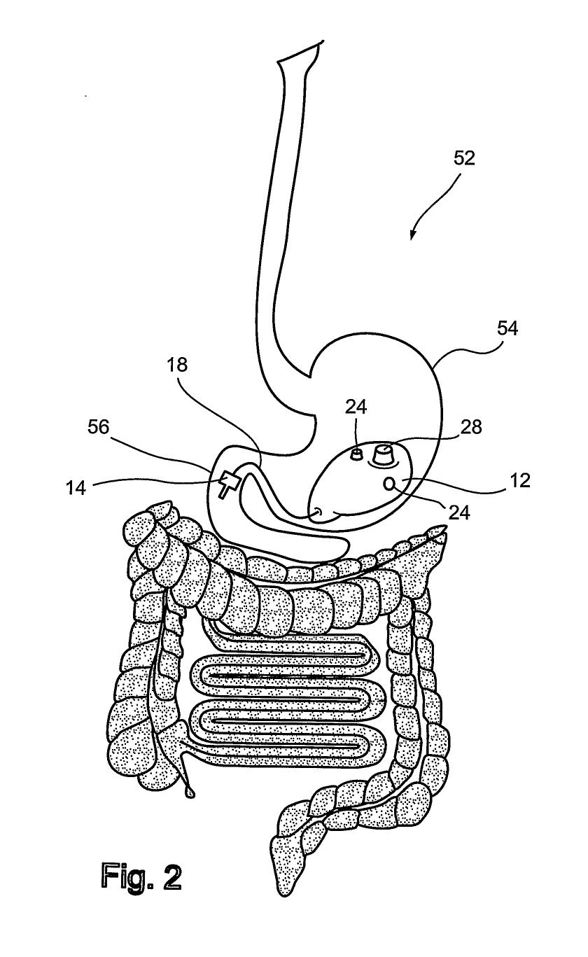

In FIG. 2, device 10 is depicted deployed in a gastrointestinal tract 52 of a person suffering from an eating disorder, where reservoir 12, deployed in stomach 54 is in fluid communication with spray head 14 located in the descending portion of duodenum 56 through feeder pipe 18.

Prior to the depicted in FIG. 2, device 10, when space-filling chambers 22 and composition chamber 26 are empty, is placed in stomach 54 with the help of a gastroscope. Spray head 14 is passed through the pyloric sphincter and deployed in duodenum 56. Using a gastroscope, reservoir 12 is deployed in stomach 54 by pumping saline solution into space-filling chambers 22 through ports 24 to a desired extent (e.g., 150 ml in each chamber 22) which is dependent, in part on the size of stomach 54 and in part to ensure that spray head 14 remains in duodenum 56.

Using a gastroscope, composition chamber 26 is charged (e.g., 100 ml) through port 28 with a sprayable pharmaceutical composition comprising an active agent suitable for treating an eating disorder. As discussed in the PCT patent application published as WO 2006/035446 of the Applicant, suitable active agents include but are not limited to satiety agents (e.g., CCK, CCK receptor agonists, PYYs, GLP-1, and oxyntomudulin and analogs thereof and derivatives thereof) and anti-food absorption drugs such as lipase inhibitors. Significantly, suitable active agents include peptides and hormones that are challenging for gastrointestinal administration.

Once deployed, device 10 is configured to automatically administer a dose of the active agent to the subject, when necessary, by spraying the sprayable pharmaceutical composition through sprayer 34 of spray head 14 at the luminal wall of duodenum 56.

The subject in which device 10 is deployed goes about life in the usual way. When the subject becomes hungry or begins to ingest food, stomach 54 begins to contract, a physiological change that is automatically detected by event detector 48. The fact of detection of the event is transmitted by event detector 48 to controller 40 of actuator 38.

Controller 40 triggers pressure generator 30 by allowing power from power storage unit 44 to pass to pressure generator 30 for a specified period of time as determined by timer 42. The dose of active agent administered is determined by the specified period of time.

Pressure generator 30 pumps sprayable composition from reservoir 26 through feeder pipe 18 into sprayer 34. The pressure at which pressure generator 30 pumps the sprayable composition into sprayer 34 forces the sprayable composition out through nozzles 32 at the luminal wall of duodenum 56. The active agent in the sprayable composition interacts with satiety chemoreceptors found on the luminal wall of duodenum 56, leading to a feeling of satiety in the subject. The feeling of satiety causes the person to eat less, thus treating the eating disorder.

It is important to note that reservoir 12 of device 10 performs at least one additional function in the framework of treating a person suffering from an eating disorder. As is clear to one skilled in the art, reservoir 12 has a stomach space-filling function in the manner of intragastric balloons known in the art for treating eating disorders to provide a feeling of fullness. In some embodiments, the magnitude of this effect is varied by adding or removing fluid from space-filling reservoirs 24 through ports 24.

Periodically, for example at fixed intervals determined by a caregiver, composition reservoir 26 is recharged with sprayable composition through port 28 using a suitably-configured gastroscope. When recharging, the nature of the composition is optionally changed, e.g., by changing the concentration of the active agent in the composition or changing the identity of the active agent.

Periodically, for example at fixed intervals determined by a caregiver, power storage unit 44 is recharged. An gastroscope bearing a suitable source of near-infrared light is placed to illuminate the photoelectric cell of power storage unit recharger 46 which converts the light to electricity and recharges power storage unit 44.

In device 10 of the present invention depicted in FIG. 1, sprayer 14 is configured to be deployed in the duodenum, and in FIG. 2 sprayer 14 is deployed in duodenum 56, as the duodenum is the preferred location for administration of the satiety agents which device 10 is designed to administer. In some embodiments of the method of the present invention, a sprayer of the present invention is deployed elsewhere, for example in the esophagus, the stomach, the antrum, antral sphincter, the fundus, the pylorus, the small intestine, the jejunum, the ileum, the large intestine, the caecum, the vermiform appendix, the colon, the ascending colon, the transverse colon, the descending colon, the sigmoid flexure or the rectum. Similarly, in some embodiments of the device of the present invention, a sprayer of the present invention is configured to be deployed elsewhere, for example in the esophagus, the stomach, the antrum, antral sphincter, the fundus, the pylorus, the small intestine, the jejunum, the ileum, the large intestine, the caecum, the vermiform appendix, the colon, the ascending colon, the transverse colon, the descending colon, the sigmoid flexure or the rectum. The specific location where a sprayer is deployed or is configured to be deployed is chosen as the preferred, the ideal or otherwise advantageous place for administration of the active agent, for example where the active agent is preferentially absorbed into the body by the gastrointestinal tract or where chemoreceptors that are affected by the active agent are located.

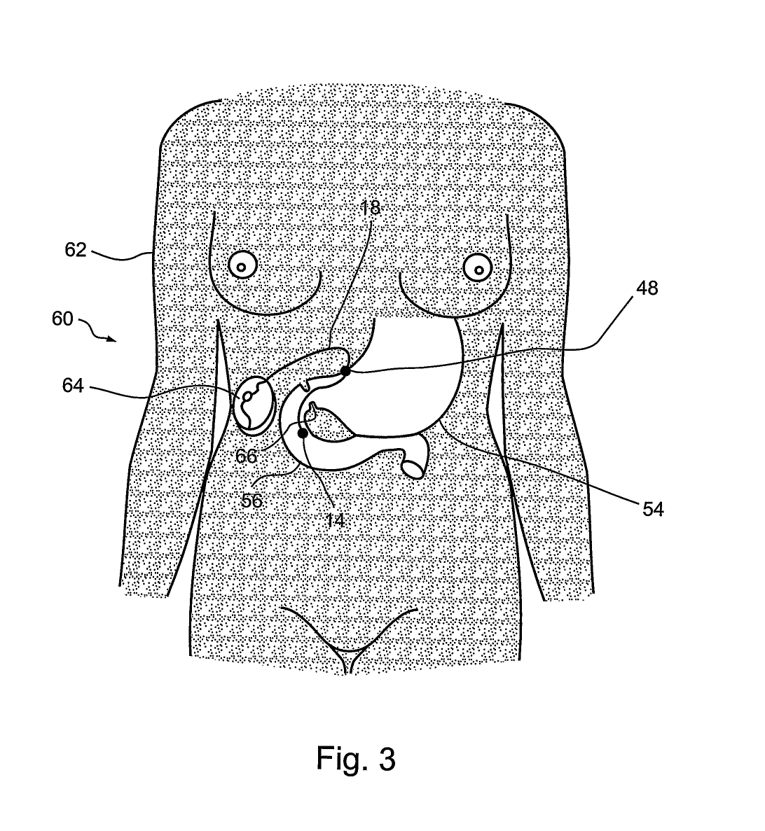

An additional embodiment of a device of the present invention configured for administration of satiety agents to the duodenum in accordance with the teachings of the present invention, device 60, is depicted in FIG. 3 deployed within a body of a subject 62.

Device 60 comprises a control unit 64 (having dimensions of roughly between 4 cm and 10 cm long, between 3 cm and 6 cm wide and between 0.5 cm and 2 cm deep) that comprises a casing in which components including a composition reservoir, a power supply unit, a pressure generator (to dispense a dose of composition held in the composition reservoir) and an actuator (e.g., a controller associated with a timer). The pressure generator in control unit 64 is in fluid communication with a spray head 14 through feeder tube 18, spray head 14 being deployed in the duodenum 56 of subject 62. Event detector 48 (for example, similar to a gastric activity detector implemented in the Tantalus™ System of Metacure NV, MetaCure N.V., Curacao, Netherlands Antilles) is physically associated with feeder tube 18 and is functionally associated with the actuator in control unit 64 by a wire running from event detector 48 to control unit 64.

Device 60 is substantially similar to device 10 discussed hereinabove with a number of notable differences.

A notable difference between device 10 and device 60, as seen in FIG. 3, is that device 60 is not simply deployed in the gastrointestinal tract of subject 62 but rather components thereof (control unit 64) are implanted subcutaneously in the body of subject 62.

Another notable difference is that unlike in device 10, in device 60 a pressure generator (a pump) is physically located with control unit 64 and not with spray head 14. The sprayer of spray head 14 of device 60 is simply a plugged tube with a plurality of nozzles arrayed about the axis of the tube and configured to direct a spray outwards.

Another notable difference is that, unlike in device 10, event detector 48 is remote from control unit 64 and is implanted inside the wall of stomach 54, allowing event detector 48 to detect and monitor gastric neural and muscle activity indicative of hunger or food ingestion. Event detector 48 serves an additional function, defining a passage through which feeder tube 18 passes through the wall of stomach 54, but preventing the passage of gastric fluids from stomach 54 into the abdomen.

The use of device 60 is substantially similar to the use of device 10 as described above and is clear to one of average skill in the art upon perusal of the description and the Figures.

In some embodiments, deployment of a device 60 of the present invention requires a number of steps, performed in any convenient order as determined by a health care professional such as a surgeon. For example, control unit 64, feeder tube 18, event detector 48 and spray head 14 are introduced into the abdominal wall and implanted subcutaneously. Using a laparoscope, feeder tube 18, event detector 48 and spray head 14 are threaded through the wall of stomach 54 and event detector 48 implanted across the wall of stomach 54. Using an intraluminal endoscope, feeder tube 18 is placed so that spray head 14 is deployed in duodenum 56 through pyloric sphincter 66. The reservoir in control unit 64 is charged (and when required, recharged) with a sprayable pharmaceutical composition comprising an active agent with the help of a transcutaneous needle as in known in the field of subcutaneously implanted devices including reservoirs (see for example the U.S. patent Application published as US 2002/0087113).

As described above for device 10, in some embodiments where a subject is treated for eating disorders, when subject 62 is hungry, sees feed or begins to consume food, event detector 48 detect physiological changes (such as electrical and mechanical activity of the stomach) indicative of the need for administering a sprayable pharmaceutical composition held in the reservoir, and transmits the fact of this need to the actuator in control unit 64. The actuator activates the pump which forces sprayable composition from the sprayable composition reservoir in control unit 64, through feeding tube 18 out through the nozzles of sprayer head 14 as a spray against the luminal walls of duodenum 56 thereby achieving a desired effect.

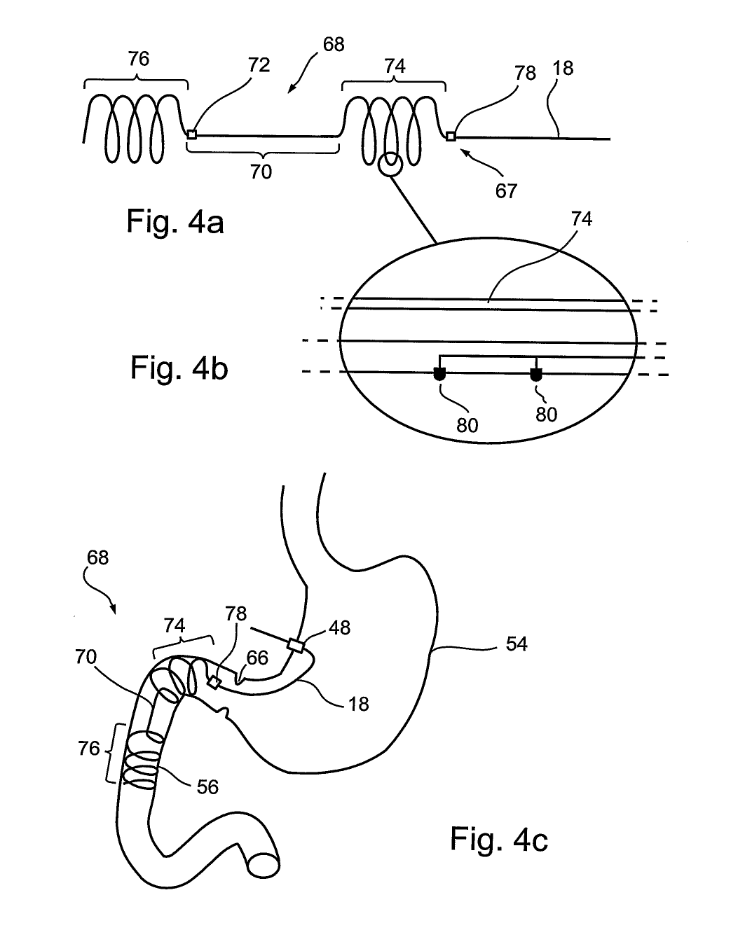

Depicted in FIGS. 4A, 4B and 4C is an embodiment of a spray head 68 of the present invention, substantially the distal end of a feeder tube 18. In FIG. 4A, spray head 68 is shown outside of the body of a subject. An enlarged cross section of the wall of spray head 68 is depicted in FIG. 4B. Spray head 68 is shown deployed in a duodenum 56 in FIG. 4C.

In spray head 68, sprayer 70 is substantially a straight length of tube delimited by an intraluminal plug 72 in fluid communication with feeder tube 18 that is flanked between a proximal anchoring section 74 and a distal anchoring section 76. Through the walls of the section of tube constituting sprayer 70 are a plurality of perforations arrayed about the axis of sprayer 70 constituting nozzles.

Spray head 68 is a flexible tube having an internal diameter of 2 mm and an outer diameter of 4 mm sections which are preshaped to assume a coiled shape to define proximal anchoring section 74 and distal anchoring section 76. To the proximal end of proximal anchoring section 74 is attached a marker 78 that is both radiopoaque and ultrasound opaque. Protruding from the outside facing walls of proximal anchoring section 74 are a plurality of gold electrodes 80 (distanced between 1 and 10 cm one from the other) that are in electrical communication with a controller (not depicted).

For deployment, a flexible but straight wire guide is inserted into the lumen of feeder tube 18, forcing feeder tube 18 to adopt a straight shape. Feeder tube 18 including spray head 68 is placed into the body (e.g., transcutaneously or through the mouth) and maneuvered past pyloric sphincter 66 into duodenum 56. Once marker 78 is just past pyloric sphincter 66, the wire guide is gradually withdrawn, allowing first distal anchoring section 76 and subsequently proximal anchoring section 74 to adopt a coil shape that presses against the luminal walls of duodenum 56. In such a way, anchoring sections 74 and 76 both position, anchor and maintain sprayer 70 in a stretched-out configuration suspended substantially parallel with the luminal walls of duodenum 56 at a distance therefrom allowing efficient spray formation and spray coverage through the nozzles of sprayer 70. Further, proximal anchoring section 74 prevents spray head 68 from being pulled out through pyloric sphincter 66 into stomach 54. Further, electrodes 80 are pressed against the luminal wall of duodenum 56. Intraluminal plug 72, e.g., a partially expandable stainless steel plug, is anchored in the appropriate location inside feeder tube 18.

The use of a sprayer 70 of a spray head 68 is, in analogy to the described above, clear to one skilled in the art upon perusal of the description herein. When needed, a pharmaceutical composition is sprayed through the nozzles of sprayer 70 against the luminal walls of duodenum 56. In addition, when needed, electricity is passed through electrodes 80 under control of a functionally associated controller to stimulate nerves in the duodenal luminal wall, for example to give the perception of satiety. Thus, spray head 68 is configured to act through two modes: stimulation of nerves with the help of electrodes 80 and administration of an active agent through nozzles of sprayer 70.

In some embodiments of spray head 68 especially configured for treating eating disorders, one or both of anchoring sections 74 and 76 are configured to press outwards with a substantial force so as to activate satiety mechanoreceptors. In such embodiments, a spray head 68 is configured to act through three modes: stimulation of nerves with the help of electrodes 80, administration of an active agent through nozzles of sprayer 70 and stimulation of mechanoreceptors by applying an outwards force to the luminal walls of a duodenum.

In some embodiments of a device of the present invention such as device 10 depicted in FIG. 1 and FIG. 2 or device 60 depicted in FIG. 3, a sprayer such as associated with spray head 14 is deployed at the specific location where administration of the sprayable composition is desired, and remains deployed at that specific location by a combination of the length of feeder tube 18 and the fact that the bulky reservoir 12 remains in place in stomach 54 (in the case of device 10) or that the event detector 48 is fixed in place in the wall of stomach 54 (in the case of device 60).

In some embodiments, a device of the present invention comprises one or more anchors, generally attached to or in the proximity of the sprayer for anchoring a deployed sprayer in place in proximity of the specific location. Anchors suitable for anchoring objects, such as a sprayer of an embodiment of a device of the present invention, in the gastrointestinal tract are well known in the art, see for example the PCT patent application published as WO2006/111961. As is known to one skilled in the art, implanted devices such as anchor are preferably implanted tension-free to prevent pain, tearing of surrounding tissue, migration and/or release of the anchor. Thus, it is preferred that an anchor anchoring a sprayer of a device of the present invention be tension free, for example, comprises a loose suture or loose stapling.

In FIGS. 1, 2 and 3, a spray head 14 may make incidental contact with luminal walls of a gastrointestinal tract in which deployed. In FIG. 4, spray head 68 which is substantially the distal end of feeder tube 18 is configured, by coiling to position and anchor sprayer 68 suspended inside the lumen of the gastrointestinal tract, far from contact with a luminal wall.

In some embodiments are generally configured to provide a spray 360° around the axis of the respective sprayer.

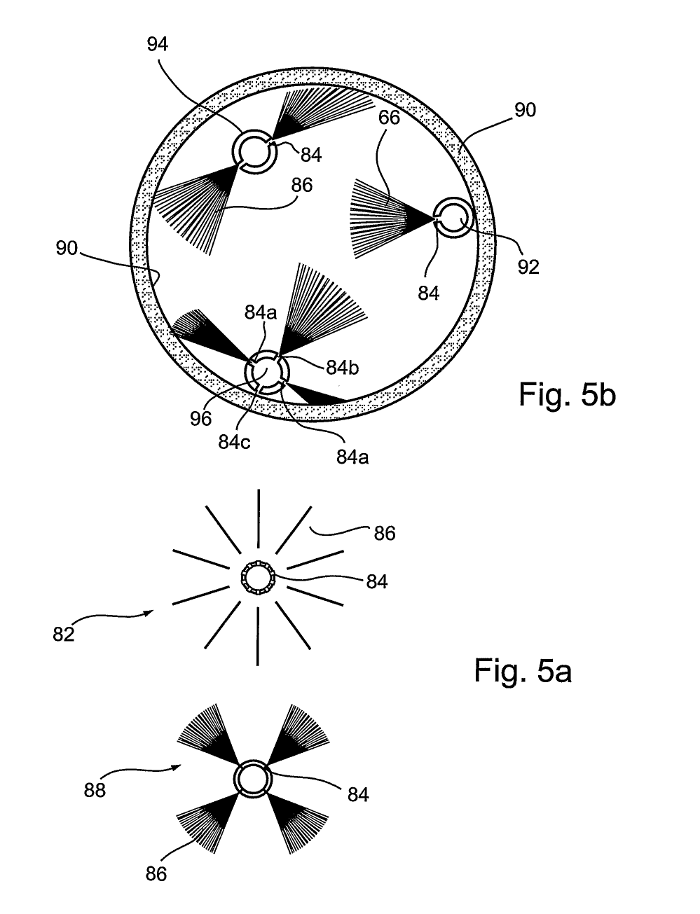

In FIG. 5A are depicted two sprayers, in cross section. Sprayer 82 includes a plurality of rows of ten nozzles 84 (one such row depicted in the cross section depicted in FIG. 5A), each row arranged about the circumference of sprayer 82 and each nozzle 84 configured to produce a narrow spray (substantially a stream) 86.

Sprayer 88 depicted in FIG. 5B includes a plurality of rows of four nozzles 84 (one such row depicted in FIG. 5B), each row arranged about the circumference of sprayer 88 and each nozzle 84 configured to produce a wide spray (approximately an arc of 60°) 86.

In some embodiments, a portion of a sprayer contacts a luminal wall, for example as a result of configuration for anchoring or deploying. For example, in a non-depicted embodiment similar to spray head 68 depicted in FIG. 4, a sprayer, substantially a plurality of nozzles is a part of one or both the anchoring sections 74 or 76 that are coiled and press against a luminal wall of a gastrointestinal tract. In such embodiments, it is generally preferred that the sprayer be configured to spray over a relatively large area of the luminal wall, for example by an arrangement of nozzles configured and arranged to spray at parts of the luminal wall both near and far from where the sprayer contacts the luminal wall.

For example, in FIG. 5B, three sprayers 92, 94 and 96 are depicted pressed against a luminal wall 90 of a gastrointestinal tract, all in cross section.

In FIG. 5B, the depicted cross section of sprayer 92 includes a single nozzle 84 with an arc of approximately 60° towards a portion of luminal wall 90 opposite the part of luminal wall 90 that sprayer 92 contacts.

In FIG. 5B, the depicted cross section of sprayer 94 includes two nozzles 84 each with an arc of approximately 45° directed towards a portion of luminal wall 90 close to the part of luminal wall 90 that sprayer 94 contacts.

In FIG. 5B, the depicted cross section of sprayer 96 includes four nozzles 84 each with an arc of approximately 45° directed so that two nozzles 84a are directed towards a near portion of luminal wall 90, one nozzle 84b is directed towards a distant portion of luminal wall 90, and one nozzle 84c is blocked.

Generally, it is desired that a pharmaceutical composition administered in accordance with the teachings of the present invention be administered to a relatively large area of the luminal wall of the gastrointestinal tract. To this end, in some embodiments a sprayer of a device of the present invention is configured to spray a length of no less than 1 cm of a luminal wall in which the sprayer is deployed, for example by including a plurality of nozzles along a length of the sprayer or by providing nozzles having a relatively large arc with respect to the length of the gastrointestinal tract. In some embodiments the length is of no less than 2 cm, of no less than 3 cm and even of no less than 4 cm.

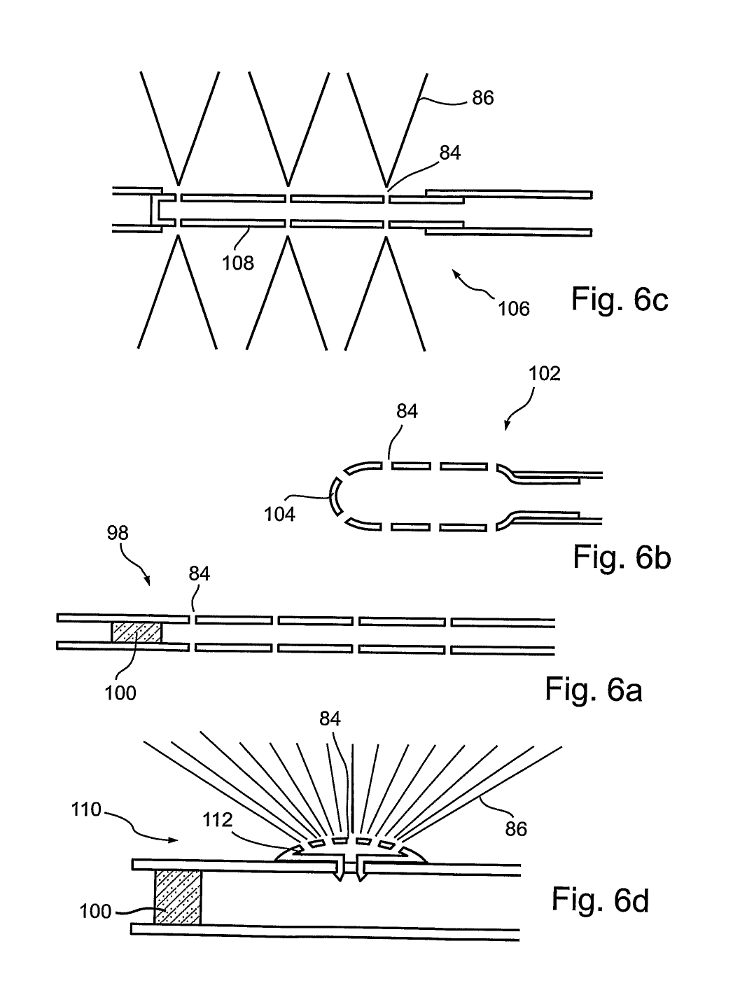

Some embodiments of sprayers suitable for implementing the teachings of the present invention are depicted in FIGS. 6A, 6B, 6C and 6D in side cross section.

In FIG. 6A, sprayer 98 is substantially a portion of the distal end of a feeder tube wherein a plug 100 inside the feeder tube defines the distal end of sprayer 98 and nozzles 84 are substantially holes, perforations, slits and the like through the wall of the tube, substantially as described in FIG. 3 and FIG. 4.

In FIG. 6B, sprayer 102 is substantially a perforated plug 104 capping the end of a feeder tube, comprising a plurality of nozzles 84. In FIG. 6C, sprayer 106 is substantially a perforated closed-end tube 108 attached to end of a feeder tube, comprising a plurality of nozzles 84.

In FIG. 6D, sprayer 110 comprises one or more spray heads 112 including a plurality of nozzles 84 placed through the wall of the feeder tube.

It is seen that each of the sprayers depicted in FIG. 6 is configured to spray a relatively significant length of a luminal wall in which deployed.

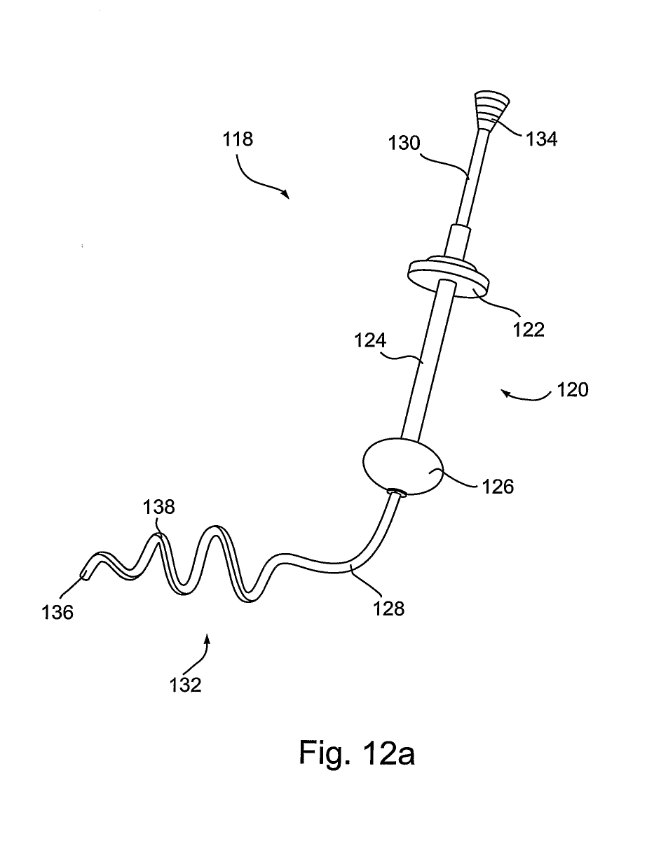

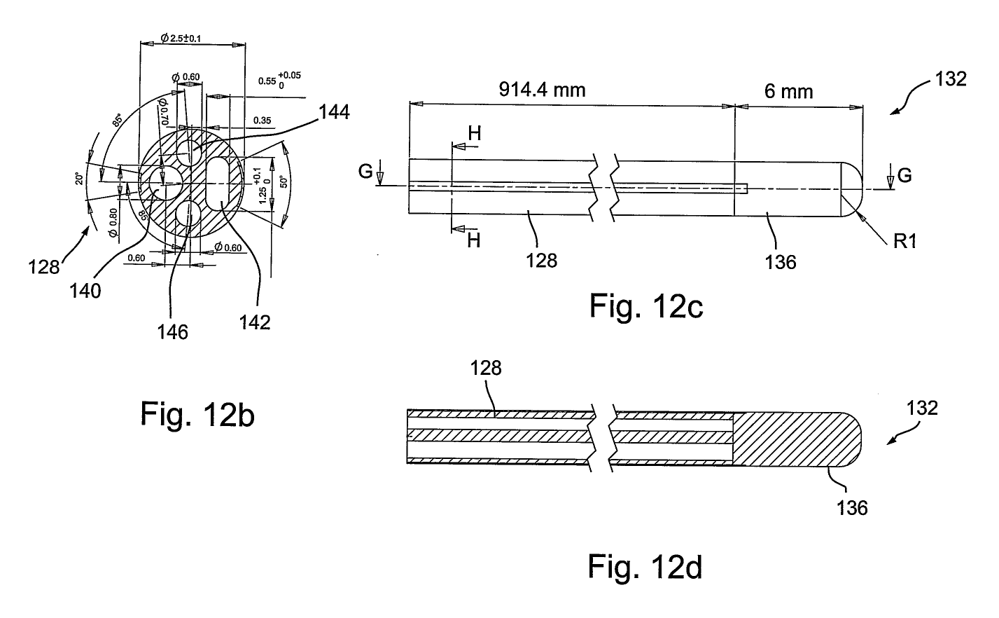

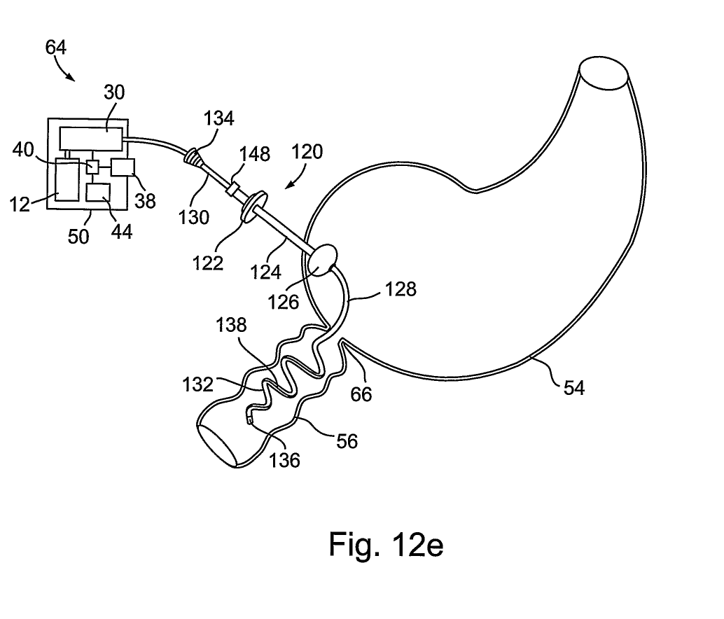

In FIGS. 12A-12E, an additional embodiment of a device useful for administering a sprayable composition to a luminal wall of the gastrointestinal tract (specifically the duodenum, specifically the superior portion of the duodenum) is depicted, device 118. In FIG. 12A, device 118 is depicted fully assembled and associated with a gastrostomy tube 120

Gastrostomy tube 120 is a standard commercially available gastrostomy tube (e.g., MicTM-“G” available from Medical Innovations Corporation, a division of Ballard Medical Products, Draper, Utah, USA) including an external button 122, a transabdominal tube 124 and an intragastric retainer balloon 126.

In FIG. 12A, it is seen that device 118 comprises a tubular body 128 having a proximal end 130 and a coiled distal end 132. Tubular body 128 has structural features so as to be configured, amongst others, as a feeder tube, a sprayer and an anchor to maintain the sprayer portion properly positioned in the duodenum of a subject.

Proximal end 130 is provided with a connector 134 allowing connection of tubular body 128 to a pressure generator (such as a pump) and a composition reservoir.

Distal end 132 ending with distal tip 136 has a conical coil shape so as to have an increased-diameter relative to the rest of tubular body 128. Coiled distal end 132 is configured to function as a sprayer: on the outer surface of distal end 132 is a slit 138 that functions as a spray orifice through which sprayable composition is forced out towards the luminal wall of a duodenum in which distal end 132 is deployed. As is discussed in detail below, slit 138 is configured to function as a valve, allowing a sprayable composition to spray outwards from tubular body 128 but substantially preventing passage of fluids into tubular body 128.

Tubular body 128 is substantially a 916 mm long by 2.5 mm diameter flexible tube of extruded Pebax 60 resin polymerized together with 20% barium sulfate. Passing coaxially through tubular body 128 are four parallel lumina. In FIG. 12B, a radial cross-section near distal end 132 of tubular body 128, is seen the arrangement of a 0.8 mm diameter active agent lumen 140, a 0.55 mm wide by 1.25 mm high rounded-rectangle lumen 142 for accepting a Nitinol strip, and two 0.6 mm diameter round electrode guiding lumina 144 and 146.

In FIGS. 12C and 12D, axial cross sections of tubular body 128 are depicted. It is seen that a 6 mm long rounded distal cap of soft polymerized Pebax 30D is secured as tip 136 of distal end 132 of tubular body 128.

For assembly, a 914 mm long, 1 mm wide and 0.4 mm thick strip of Nitinol (not depicted) formed so that a distal end thereof adopts the desired shape of a 4 cm long conical coil having 3.5 loops is passed through rounded-rectangle lumen 142. The Nitinol strip forces distal end 132 of tubular body 128 to adopt the conical coiled shape depicted in FIG. 12A where active agent lumen 140 is on the outside of the coil. In such a way, coiled distal end 132 of tubular body 128 is configured to function as an expandable increased-diameter feature.

On the outer face of coiled distal end 132 of tubular body 128, a sharp knife is used to make slice 138 coaxial to tubular body 128 through the wall of tubular body 128 to active agent lumen 140 forming an orifice. In such a way, coiled distal end 132 is configured as a sprayer, where slice 138 functions as a valve allowing a sprayable composition to be forced out as a spray, but preventing entry of liquids back into active agent lumen 140, sees below.

In the art of gastrointestinal surgery, percutaneous endoscopic gastrostomy is used to endoscopically deploy a gastrostomy tube through the abdominal wall to provide a passage from the outside of the body into the stomach cavity.

In an embodiment for deploying device 118, gastrostomy tube 120 is deployed in the usual way so that external button 122 contacts the skin of a subject and intragastric retainer balloon 126 is inflated inside the stomach cavity of the subject so that bodily tissue is clamped between external button 122 and intragastric retainer balloon 126 while transabdominal tube 124 defines a direct channel from outside the body to the stomach cavity.

Device 118 is threaded through a delivery tube (not depicted, but e.g., 3 mm inner diameter, 4 mm outer diameter braided stainless steel flexible tube lined with polytetrafluorethylene and covered with a Pebax® polymer sleeve) forcing coiled distal end 132 into a straight conformation. While encased in the delivery tube, device 118 is threaded, distal tip 136 first, through transabdominal tube 124 of gastrostomy tube 120 into the cavity of a stomach of a subject. Under guidance of and with the help of a gastroscope, distal tip 136 is guided through the pyloric sphincter and into the duodenum. The delivery tube is carefully withdrawn while device 118 is pushed forward. As distal end 132 emerges from the delivery tube, distal end 132 expands into the coiled conformation. Ultimately, distal end 132 is completely coiled and the delivery tube entirely withdrawn from gastrostomy tube 120. A retaining clip 148 is secured around tubular body 128 and against the outer side of external button 122, to act together with tubular body 128 as an anchor so that distal end 132 is maintained in the superior portion of the duodenum.

In FIG. 12E, device 118 is depicted properly deployed in the gastrointestinal tract of a subject, where the abdominal wall and other abdominal tissue are not depicted. In FIG. 12E is seen how external button 122 contacts the skin and inflated intragastric retainer balloon 126 contacts the inner surface of stomach 54 so as to clamp bodily tissue therebetween so that transabdominal tube 124 defines a direct channel from outside the body to the cavity of a stomach 54. Connector 134 and proximal end 130 of tubular body 128 are located outside the body of the subject. Tubular body 128 passes through pyloric sphincter 66 while coiled distal end 132 of tubular body 128 is located in the superior portion of duodenum 56.

Coiled distal end 132 of tubular body 128 functions as a sprayer and also as an increased-diameter portion so as to prevent distal end 132 from moving outwards through pyloric sphincter 66. Clip 148 together with the length of tubular body 128 act as an anchor to maintain coiled distal end 132 in the superior portion of duodenum 56 about 1 cm from pyloric sphincter 66.

For use, a control unit 64 is mated to connector 134. Control unit 64 includes a pressure generator 30, a composition reservoir 12, an actuator 38 (a manually operable switch), a controller 40 and a power storage unit 44 and is configured to be deployed outside of the body of the subject. Control unit 64 is similar to the control unit of the DuoDopa® device (Solvay Pharmaceuticals GmbH, Hannover, Germany).

When actuator 38 is triggered, controller 40 actuates pressure generator 30 to pump sprayable composition (e.g., a pharmaceutical composition including an active agent) from composition reservoir 12, past connector 134, into active agent lumen 140 of tubular body 128. The pressure forces the sprayable composition through slit 138 as an outwardly oriented sheet-like spray, administering the composition in accordance with embodiments of the invention. After sufficient time has passed for a desired dose to have been administered, controller 40 stops pressure generator 30 so that composition is no longer forced through slit 138 and the pressure in active agent lumen 140 is reduced. When the pressure is reduced, the elasticity of the walls of body 128 forces slit 138 closed, preventing entry of materials into active agent lumen 140.

In device 118, a prior art gastrostomy tube 120 is used to define a passage through which tubular body 128 of device 118 passes into the body of the subject. In some embodiments, a device body such as tubular body 128 is fashioned having features (e.g., integrally formed with or attached to) of a gastrostomy tube 120 such as external button 122 and intragastric retainer balloon 126 rendering a separate transabdominal tube 124 unnecessary. In some embodiments, a different type of gastrostomy tube or functionally equivalent component is used.

In some embodiments, instead of a control unit such as 54 located outside of the body of the subject, a control unit is configured to be implantable inside the body (for example subcutaneously, in the abdominal cavity) or inside the gastrointestinal tract, as discussed hererinabove and hereinbelow.

In some embodiments of a device of the present invention such as device 10 depicted in FIG. 1 and FIG. 2, a reservoir such as reservoir 12 is deployed in the stomach. Deploying in the stomach is simple as the stomach is relatively large and flexible allowing a relatively large reservoir to be deployed therein with no ill effects. The stomach is easily accessible through the esophagus with a gastroscope, allowing relatively simple deployment and maintenance (such as recharging of a composition reservoir or a power supply unit) of the device. Further, in some embodiments contractions of the muscular stomach assist in driving sprayable composition from a composition reservoir to the sprayer. Further, as discussed above for device 10, in some embodiments a reservoir deployed in the stomach acts as an intragastric space-filling balloon, a property useful as an adjunct when treating certain ailments.

Despite the advantages of deploying the reservoir of a device of the present invention in the stomach, in some embodiments a reservoir is deployed elsewhere in the gastrointestinal tract, for example closer to the specific location where the sprayable composition is to be sprayed. Although in some embodiments a reservoir is deployed anywhere in the gastrointestinal tract, preferred locations for deploying a reservoir of a device of the present invention include the large intestine, the caecum, the vermiform appendix, the colon, the ascending colon, the transverse colon, the descending colon, the sigmoid flexure or the rectum.

In some embodiments of a device of the present invention such as device 60 depicted in FIG. 3, a reservoir such as reservoir 12 is deployed and anchored subcutaneously. Subcutaneous implantation of composition holding reservoirs is well-known in the art and allows for simple recharging of the reservoir with a pharmaceutical composition including an active agent when necessary. Despite the advantages of deploying a device of the present invention subcutaneously, some embodiments of the present invention include a reservoir deployed elsewhere in the body.

In some embodiments, an actuator is configured to actuate the pressure generator so as to dispense a specified dose of sprayable composition. In some embodiments such as device 10 depicted in FIG. 1 and FIG. 2 or device 60 depicted in FIG. 3, the dispensed dose is a fixed dose determined by the time which actuator 38 comprising controller 40 and timer 42 trigger pressure generator 30 to produce a spray by providing power from power storage unit 44. In some embodiments, a device of the present invention comprises a dosage adjusting mechanism functionally associated with the actuator and/or with the pressure generator that allows the dosage to be changed or adjusted while the device is deployed in the gastrointestinal tract. For example, in some embodiments a dosage adjustment mechanism is functionally associated with the actuator and functions by changing the length of time which an associated pressure generator is actuated thus changing the dose. For example, in some embodiments, a device of the present invention comprises a wireless receiver functionally associated with a controller and the controller is configured to accept commands to change the length of time that the pressure generator is actuated. For example, in some embodiments a dosage adjustment mechanism is functionally associated with the pressure generator and functions by changing the volume of a composition that is sprayed per unit time, for example by changing or adjusting the stroke volume of a piston or like device. In some embodiments, the dosage adjusting mechanism is mechanical and is performed endoscopically, for example by turning a stroke-volume adjusting screw.

As noted above, some embodiments of the present invention include an event detector functionally associated with an actuator so that as a result of detection of an event of significance for administration of the active agent, the event detector triggers the actuator to administer the active agent. In device 10 depicted in FIG. 1 and FIG. 2, event detector 48 is a pressure sensor configured to detect a gastric contraction event associated with hunger or food ingestion. In device 60 depicted in FIG. 3, event detector 48 is an electrical activity sensor configured to detect an electrical activity event in the wall of stomach 54 associated with hunger or food ingestion. One skilled in the art, upon perusal of the disclosure herein, is able to select and modify any of the different event detectors and sensors known in the art to implement of the teachings of the present invention, for example the electrode-comprising detectors disclosed in the PCT patent application published as WO 2006/035446 of the Applicant, gastric activity detectors such implemented in the Tantalus™ System (Metacure NV, MetaCure N.V., Curacao, Netherlands Antilles), or event detectors described in the U.S. patent application published as US 2005/0096637, pressure sensors (e.g., Chronicle® Medtronic, Inc., Minneapolis, Minn., USA), muscle activity sensors such as described in the U.S. patent application published as US 2004/0220633 or available from Delsys Inc. (Boston, Mass., USA), pH sensors (e.g., Bravo®, Medtronic, Inc., Minneapolis, Minn., USA).

In some embodiments, an event detector is in wired communication with the actuator. In some embodiments, an event detector is in wireless communication (e.g., radio frequency or near-infrared communication) with the actuator.

In device 10 depicted in FIG. 1 and FIG. 2 and in device 60 depicted in FIG. 3, actuator 38 comprising controller 40 and timer 42 is configured to trigger pressure generator 14 to begin administering a dose of sprayable composition upon detection of an event by event detector 48. In some embodiments, actuator 38 is functionally associated with a timer and is configured to begin administering a dose of sprayable composition a specified period of time after detection of an event as determined by the timer. In some embodiments, the specified period of time can be changed or adjusted while the device is deployed in the gastrointestinal tract. For example, in some embodiments, a device of the present invention comprises a wireless receiver functionally associated with a controller and the controller is configured to accept commands to change a delay between detection of an event and initiation of administration of the sprayable composition.

In some embodiments, administration of a composition is event-driven, that is subsequently to detection of an event that is of significance for administration of the active agent whether manually (by the subject or by a caregiver) or automatically (by an event detector such as event detector 48 of device 10 depicted in FIG. 1 and FIG. 2 or device 60 in FIG. 3), a sprayable composition is administered in accordance with the teachings of the present invention. In some embodiments, administration of a composition is periodic and administration of a composition in accordance with the teachings of the present invention is initiated according to a periodic schedule, whether manually by the subject or care giver, or automatically with a device configured for such.

In some embodiments, a device of the present invention is configured for periodic administration of a composition by functionally associating an actuator with a timer, and the actuator is configured to periodically trigger the pressure generator with reference to the timer. In some embodiments, the administration protocol (how often a dose is administered) is specified. In some embodiments, the device is configured to allow the administration protocol to be changed or adjusted while the device is deployed in the gastrointestinal tract. For example, in some embodiments, a device of the present invention comprises a wireless receiver functionally associated with a controller and the controller is configured to accept commands to change the frequency or timing or other parameters of the administration protocol.

In device 10 depicted in FIG. 1 and FIG. 2 and device 60 depicted in FIG. 3 pressure generator 30 comprises an electrical pump to spray a sprayable composition in accordance with the teachings of the present invention. Other suitable devices useful as pressure generators to implement the teachings of the present invention include such devices as spring-powered pressure generators, gas-pressure powered pressure generators (comprising, for example, a compressed gas reservoir and a valve) and syringes.

As described above, device 10 depicted in FIG. 1 and in FIG. 2 is provided with a power supply unit including power storage unit 44 (a battery) and power storage unit charger 46. In some embodiments a device of the present invention is provided with a non-rechargeable power storage unit. In some embodiments, a power supply unit of a device of the present invention includes a power generation unit, e.g. a kinetic power generation unit, similar to the described in U.S. Pat. No. 6,154,422 that converts motions (such as shaking, moving or jostling) of an object with which a kinetic power generation unit is associated to electrical power.

As described above, device 10 depicted in FIG. 1 and FIG. 2 and device 60 depicted in FIG. 3 are configured to administer an active agent through a sprayer deployed in the gastrointestinal tract when necessary. In some embodiments, administration of an active agent when necessary is optionally accompanied by additional modes and methods of treatment that are used continuously, simultaneously with or in parallel with the administration of the active agent. For example, in analogy to the discussed in the PCT patent application published as WO 2006/035446 of the Applicant, in some embodiments of the invention useful for treating eating disorders, additional modes and methods of treatment include direct vagal nerve stimulation using an implanted electrode or other forms of gastric stimulation such as implemented in the Tantalus™ System (Metacure NV, MetaCure N.V., Curacao, Netherlands Antilles).

Described above are some embodiments of the present invention used to treat conditions relating to eating disorders such as obesity, where a satiety agent is administered by spraying directly to interact with chemoreceptors lining the duodenal lumen in response to detection of a physiological change (stomach contraction, stomach wall electrical activity) indicative of an event (hunger, food ingestion) of significance to the need of administration of the satiety agent. In some embodiments, the teachings of the present invention are used for treating other conditions.

In some embodiments, the teachings of the present invention are used to administer an active agent that does not affect chemoreceptors at or near the luminal walls but rather is absorbed through the luminal wall and into the blood stream.

For example, in an embodiment, the teachings of the present invention are used to treat subjects suffering from a cardiovascular pathology and specifically to administer an active agent when an event such as an anginal attack or arrhythmia is detected. For example, the electrical activity of the heart is monitored in real time using a pacemaker such as a Model 1298 Insignia (Guidant Corporation a part of Boston Scientific, Inc. Natick, Mass., USA) provided with wireless transmitter to a device of the present invention provided with an actuator functionally associated with a wireless receiver.

When the actuator receives an indication that an anginal attack or arrhythmia has been detected, an anti-arrhythmic or anti-anginal material as an active agent is administered through a sprayer deployed in the ileum. The active agent is rapidly absorbed, giving an almost immediate effect in response to the anginal attack or the arrhythmia. Suitable active agents include adenosine or nitrates (e.g., isosorbide dinitrate, isosorbide mononitrate, and nitroglycerin).

For example, in some embodiments, the teachings of the present invention are used to treat subjects suffering from asthma and specifically to administer an active agent when an event such as an asthmatic attack is detected. For example, a person suffering from asthma is provided with a portable trigger (e.g., a wrist borne “panic button”) provided with wireless transmitter to a device of the present invention provided with an actuator functionally associated with a wireless receiver.

When the actuator receives an indication that an asthma attack has been detected for example, the “panic button” has been pressed, an anti-asthma material as an active agent is administered through a sprayer deployed in the ileum. The active agent is rapidly absorbed, giving almost immediate relief from the asthma. Suitable active agents include systemic bronchial activators such as salbutamol.

For example, in some embodiments, the teachings of the present invention are used to treat subjects suffering from an allergy and specifically to administer an active agent when an event such as an allergic reaction, especially as systemic allergic reaction such as anaphylaxis is detected. For example, a person suffering from an allergy is provided with a portable trigger (e.g., a wrist borne “panic button”) provided with wireless transmitter to a device of the present invention provided with an actuator functionally associated with a wireless receiver.

When the actuator receives an indication that an allergic reaction has been detected for example, the “panic button” has been pressed, an anti-allergic or anti-anaphylactic material as an active agent is administered through a sprayer deployed in the ileum. The active agent is rapidly absorbed, giving almost immediate relief from the allergic reactions. Suitable active agents include epinephrine.

In some embodiments, the teachings of the present invention are used to periodically administer an active agent for maintenance or prophylaxis rather than in response to detection of an event.

For example, in an embodiment, the teachings of the present invention are used to treat subjects suffering from inflammatory bowel disease and specifically to topically treat inflammation in the gastrointestinal tract by administering a sprayable composition including an anti-inflammatory such as acyl salicylic acid. In an embodiment of the invention, a reservoir of a device of the present invention is charged with a sprayable acyl salicylic acid composition and a timer of a control unit is functionally associated with an actuator to periodically trigger the actuator to administer one or more daily doses of the composition. The sprayer is deployed in the terminal ileum and preferably configured to spray directly at an inflamed region.

The use of a device of the present invention to implement the method of the present invention has many advantages over prior art oral administration including that patient compliance is absolute even for complex administration regimens so that dosage is carefully controlled and can occur multiple times over day, ensuring a relatively constant plasma concentration and where an active agent administered comprises multiple materials.

For example, in an embodiment, the teachings of the present invention are used to treat subjects suffering from an infection of helicobacter pylori and specifically to topically treat ulcers in the gastrointestinal tract (both gastric ulcers and duodenal ulcers) by administering a sprayable composition including an anti-ulcer active agent. In an embodiment of the invention, a reservoir of a device of the present invention is charged with a sprayable anti-ulcer composition and a timer of a control unit is functionally associated with an actuator to periodically trigger the actuator to administer one or more daily doses of the composition. The sprayer is deployed in the vicinity of ulcers and preferably to spray directly at an ulcer.

In some embodiments, a composition includes an antibiotic, for example Amoxicillin, Clarithromycin, Metronidazole or Tetracycline. However, it is known that treatment of a helicobacter pylori infection is best performed by administration of an active agent comprising two, three or even four different active materials.

In some embodiments, a composition includes two materials, such as an antibiotic and a proton pump inhibitor (PPI), e.g., Esomeprazole, Lansoprazole, Omeprazole, Patoprazole or Rabeprazole. Particularly suitable are combinations of Clarithromycin with a PPI.

In some embodiments, a composition includes three materials, such as Clarithromycin with Metronidazole and a PPI; Amoxicillin with Clarithromycin and a PPI; Amoxicillin with Metronidazole and a PPI; or Tetracycline with Metronidazole and a cytoprotective agent (e.g. Bismuth subsalicylate or preferably sucralfate).

In some embodiments, a composition includes four materials, such as a cytoprotective agent (e.g. sucralfate or preferably Bismuth subsalicylate) with Metronidazole and Tetracycline and a H2Blocker (e.g., Cimetidine, Famotidine, Nizatidine and Ranitidine); a cytoprotective agent (e.g. sucralfate or preferably Bismuth subsalicylate) with Metronidazole and Amoxicillin and a H2Blocker; a cytoprotective agent (e.g. sucralfate or preferably Bismuth subsalicylate) with Metronidazole and Tetracycline and a PPI and a cytoprotective agent (e.g. sucralfate or preferably Bismuth subsalicylate) with Metronidazole and Clarithromycin and a PPI.

It is expected that during the life of this patent many relevant detectors, active entities and useful physical components such as pumps will be developed and the scope of the patent is intended to include all such a priori.

Additional objects, advantages, and novel features of the present invention will become apparent to one ordinarily skilled in the art upon examination of the following examples, which are not intended to be limiting. Additionally, each of the various some embodiments and aspects of the present invention as delineated hereinabove and as claimed in the claims section below finds experimental support in the following examples.

### EXAMPLES

Reference is now made to the following examples which together with the above description illustrate the invention in a non limiting fashion.

A study of the feasibility of the teachings of the present invention was performed in the context of treating the obesity using CCK-8.

CCK-8 is an 8 amino acid long fully active peptide analogue of CCK (the enteric hormone cholecystokinin). CCK is secreted from duodenal cells in response to the presence of food in the duodenum. The primary effects of CCK secretion are the release of bile from the gallbladder into the gut lumen and increased pancreatic secretion. CCK is also an enteric satiety factor. It is known that intravenous administration of CCK causes termination of food consumption by delaying gastric emptying and inducing a feeling of satiety. It is known that the CCK-induced bile release from the gallbladder and increased pancreatic secretions are mediated through CCKa receptors located on vagal nerve endings in the duodenal wall. It has been hypothesized that administration of CCK or an analogue thereof to the duodenal wall would lead to activation of CCKa receptors on the duodenal wall, leading to a feeling of satiety and meal termination which would reduce the amount of food eaten.

Obese volunteer subjects having a BMI greater than 35 kg cm⁻² were selected. The subjects were all between 18 and 50 years old, had no history of heart disease (MI, arrhythmia), no history of kidney disease or proteinurea (serum creatinin <1.5 mg dl⁻¹), no history of peptic ulcers, intestinal disease, intestinal surgery, pancreatitis, gall stones or liver disease (SGOT, SGPT, Alk Phos <X3), no endocrine dysfunction, no diabetes mellitus, no active medical disease except hypertension, were taking no medication except thyroid supplements and medications for controlling blood pressure and blood lipids.

The subjects were fitted with intragastric balloons and various types of nasoduodenal tubes deployed in the duodenum. No less than two weeks after fitting with the intragastric balloon, those subjects that showed no complications resulting from the nasoduodenal tube (e.g., vomiting) were considered as having enrolled in the study, that day being designated as the enrollment day. In a first three week period the subjects were acclimatized to the study, learning to eat with the nasoduodenal tube and becoming familiar with the study methods. Various parameters of CCK-8 administration were tested including frequency, dose, the nature of the pharmaceutical composition including CCK-8 and the nature of the nasoduodenal tube. It was unexpectedly found that a fast and forceful introduction of CCK-8 containing composition through nasoduodenal tubes configured with holes that lead to spraying of the composition towards the luminal walls of the duodenum was, all things being equal, significantly more efficient in eliciting a response attributable to CCK-8 absorption than a lower rate introduction where the composition flowed or dripped into the duodenum.

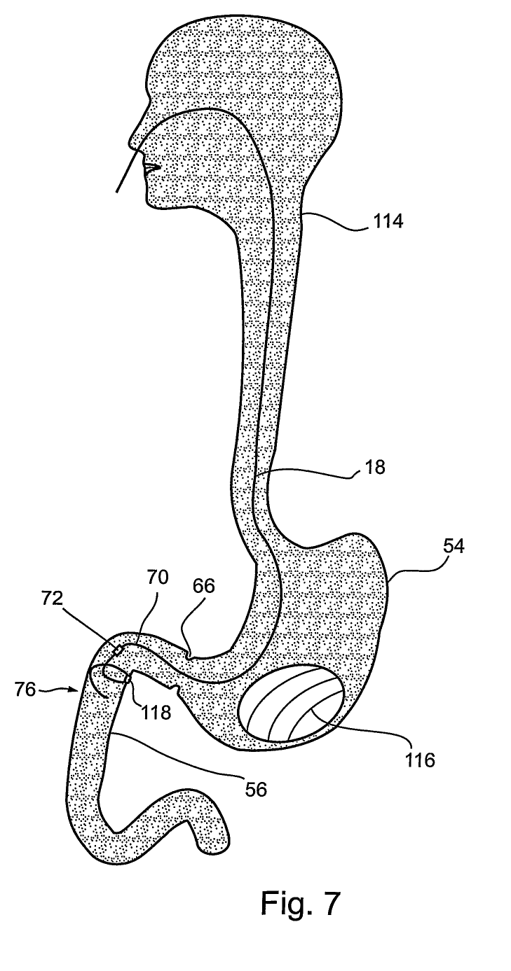

An administration device (schematically depicted in FIG. 7) in accordance with the teachings of the present invention was made. A length of “pig tail” nasobiliary tube (Cook Nasal Biliary Drainage Sets, ENBD-6-Liguory, GPN G21725) was taken as a feeder tube 18. Approximately 40 cm from the distal end, tube 18 was blocked with a stainless steel 316 LVM plug 72. Tube 18 was perforated (on the proximal side of and close to plug 72) with three rows of three nozzles each constituting a sprayer 70, each row parallel to the tube axis and 120° from the other rows, each nozzle substantially a 0.5 mm diameter perforation in the tube wall directed perpendicularly outwards from the axis of tube 18.

The subjects 114 were fitted with an intragastric balloon 116 followed by endoscopic placement of the administration device so that sprayer 70 was situated in the superior portion of the duodenum 56. Placement of a sprayer 70 in the proper location in duodenum 56 was assisted by using plug 72 as a marker apparent in fluoroscopy and sonoscopy. Sprayer 70 was maintained in place by the 40 cm distal pig-tail that extended further into duodenum 56 as a distal anchoring section 76, by friction of the curly “pig tail” structure with the luminal walls of duodenum 56, and by anchoring with the use of a clip 118 (using a Resolution™ Clip Clipping Device REF 2261, Boston Scientific) to the duodenum wall.

Doses of approximately 6 ml of a sprayable composition including an amount of CCK-8 (Clinalpha Ltd.) mixed with a pharmaceutically acceptable carrier such as saline were administered over a 10 second period using a syringe attached to the proximal end of the feeding tube (intraduodenal bolus injection). The relatively high rate of administration ensured that the composition was sprayed (rather than dripped) out through the nozzles of the sprayer at the luminal wall of the duodenum where the CCK-8 of the composition interacted with receptors on the luminal wall to induce a feeling of satiety.

### Effect of CCK-8 Administration on Gallbladder Contraction

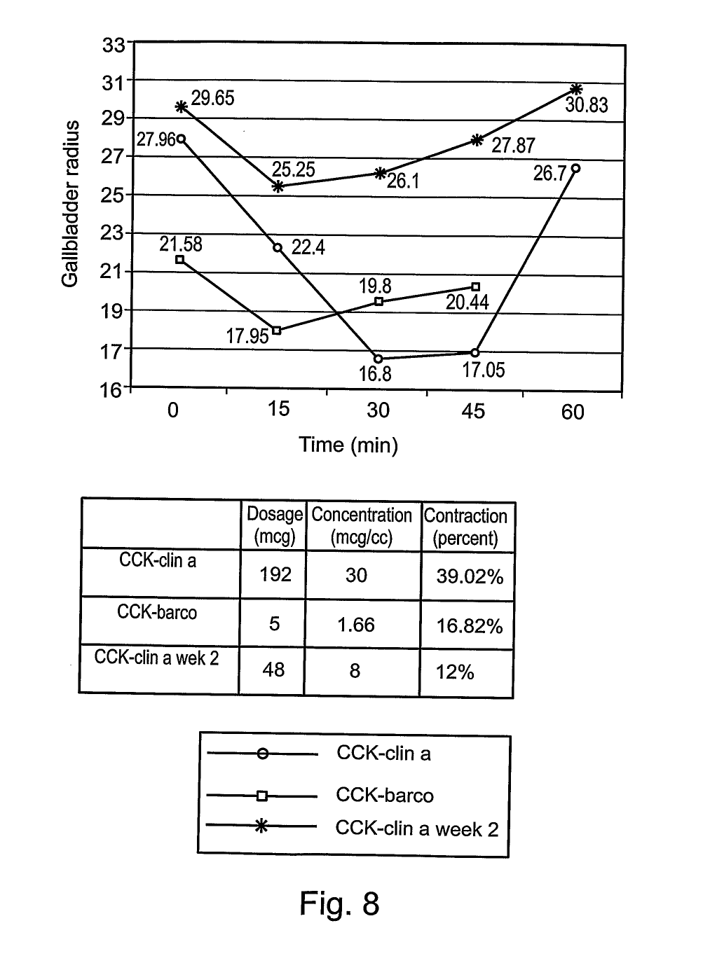

The size of the filled gallbladder of two patients in a fasting state was measured using ultrasonography. Fifteen minutes later, a dose of the sprayable composition was administered. To subject A was administered a dose of 192 μg CCK-8 (Clinalpha, Darmstadt, Germany) in a 6.4 ml volume. To subject B was administered a dose of 5 μg CCK-8 (Braco SpA, Milano, Italy) in a 3 ml volume and after two weeks 48 μg CCK-8 (Clinalpha, Darmstadt, Germany) in 6 ml. Ultrasound imaging and recording of the gallbladder was performed every 15 minutes to determine its ejection fraction. The examination was performed at 15, 30, 45 and 60 minutes or until substantial expansion of the gallbladder occurred. All images and recordings were acquired from the same angle and by the same technician using a Hitachi 8500 ultrasound device with a probe. The gallbladder volume was calculated using the dedicated software of the ultrasound device. The ejection fraction was calculated by dividing the volume of the contracted bladder by its volume at baseline (extended bladder). The results are presented in Table I and in FIG. 8.

TABLE I
Effect of administration of CCK-8 in accordance with the teachings of the
present invention on gallbladder contraction

| | Dose | Concentration | Contraction |
|---|---|---|---|
| Subject | (μg) | (μg ml⁻¹) | (%) |
| A | 192 | 30 | 39.02 |
| B (first) | 5 | 1.7 | 16.82 |
| B (second) | 48 | 8 | 12 |

Intraduodenal administration of CCK-8 in accordance with the teachings of the present invention led to a clear-cut and substantial contraction of the gallbladder. It was seen that when administered in accordance with the teachings of the present invention, CCK-8 led to up to about 39% gallbladder contraction, substantially equal to the 46.4±19.5% contraction of the gallbladder known in the art for gallbladder contraction caused by 4 μg intravenously administered CCK-8. Although the amount of CCK-8 administered was higher (192 μg as opposed to 4 μg), it must be remembered that the results were obtained in preliminary experiments. Further, it must be remembered that the non-invasive administration of an active agent in accordance with the teachings of the present invention is preferable to invasive intravenous administration, even if a larger amount of active agent must be used.

### Effect of CCK-8 Administration on Caloric Consumption, Food Consumption and Food Taken to the Plate

Six subjects from amongst the enrollees were selected for participation in the study, Table II.

TABLE II
Characteristics of subjects participating in study

| | | | Height | Weight | |
|---|---|---|---|---|---|
| | Age | sex | [cm] | [kg] | BMI |
| 1 | 43 | M | 182 | 144.5 | 44.6 |
| 2 | 47 | F | 162 | 150.5 | 58.8 |
| 3 | 48 | M | 166 | 117.0 | 40.5 |
| 4 | 34 | M | 155 | 117.0 | 49.0 |
| 5 | 34 | M | 176 | 122.4 | 39.5 |
| 6 | 42 | F | 164 | 128.0 | 47.6 |

No less than four weeks from the enrollment day of the subjects, an administration device as described above was inserted endoscopically to the six subjects. Sprayer location was evaluated twice a week by fluoroscopy. Reintroduction was performed when the sprayer was not properly located.

The six subjects stayed in the recovery department of a hotel associated with a medical center. Meals were served in a private room at the hotel restaurant in pleasant surroundings. The amount of food on a plate was weighed at the beginning and end of a meal to give an accurate estimate of food taken and consumed. Video recordings of the subjects were taken of all subjects throughout the meals.

Food weighing was performed with the plate twice before and after every meal, where one type of plate was used for breakfast and dinner and a different type of plate used for lunch. Additional weighing was performed whenever a subject took an extra portion. Drinks were calculated by glass rather than weighed.

Caloric intake was calculated in accordance with the following values: (calories per 100 gram): bread (250), cheese (100), vegetables/salad (40), egg (133), tuna (200), yoghurt (60), vegetable soup (67), meat/fish (170), boiled vegetable (40), rice/mashed potatoes (267) and fruit dessert (67).

The six subjects were served three meals a day. Breakfast and dinner were served as buffets in which subjects could choose as much food as desired. The buffets included bread, vegetables, vegetable salad, several sorts of low fat cheeses, egg, tinned tuna, yoghurt and juice or water. Lunch was a la carte and included soup and/or salad as a first course, a main course of meat or fish, rice or mashed potatoes, steamed vegetable, a fruit dessert and water or juice.

Two hours before a meal, saline as a placebo and a sprayable composition was prepared including CCK-8 as an active ingredient at the hospital pharmacy. The composition and placebo were placed in identical vials labeled only with the subject's name. The subject and the treating health-care professional were not aware which subject received a placebo and which the composition.

Five minutes prior to each meal, depending on the subject a placebo, a composition of 48.4 μg in 6 ml or of 96.8 μg in 9 ml was administered over an approximately 10 second period using a syringe attached to the proximal end of the feeding tube. The relatively high rate of administration ensured that the composition was sprayed (rather than dripped) out through the nozzles of the sprayer at the luminal wall of the duodenum where the CCK-8 of the composition interacted with receptors on the luminal wall to induce a feeling of satiety.

The weight and composition of food taken and consumed was recorded. After each meal, for each subject, the amount of food taken (grams), the amount of food eaten (grams) and amount of food eaten (calories) were calculated.

During the first week, the six subjects received placebo for a first half a week (Sunday, Monday, Tuesday) and then a CCK-8 composition for the second half week (Wednesday, Thursday, Friday). Subsequently, the six subjects were invited to participate in an additional two weeks experimental period. During the experimental period, placebo was administered for the entire first week and CCK-8 containing composition was administered for the entire second week. At each meal, the subjects were asked to complete a VAS questionnaire regarding the degree of hunger and fullness before administration, before the meal (5 minutes later) and after the meal. The VAS questionnaire included a 1-10 scale for hunger and fullness, with 3 points of verbal description (“extremely hungry”, “not hungry, not full” and “extremely full”). Nausea, discomfort or side effects (verbal description) were recorded by a coordinator.

### Numerical and Statistical Analysis

The sample size was limited to six subjects. Each subject consumed at least 10 meals with administration of CCK-8 or placebo and the effects on food consumption were compared. Based on prior work with other gastrointestinal tract hormones, a 15% decrease in caloric consumption with a standard deviation of 10% was postulated. The sample size was selected to detect such a difference.

For each subject, data on the amount consumed in each meal was characterized by two parameters meal (B, L, D) and treatment (CCK-8 or placebo). Each meal was treated individually assuming no hierarchy or connection between them and assuming no carry over effect. Sixteen meals were excluded from the final analysis, 11 due to technical problems such as tube insertion or migration, four due to protocol violations such as when a subject had a personal emergency preventing the meal from being finished.

A 3-way unbalanced ANOVA was used to determine if CCK-8 administration had any impact on food consumption was used. The model was formulated as follows:

yᵢⱼₖₗ=μ+αᵢ+βᵢ+γₖ+(αβ)ᵢⱼ+(βγ)ⱼₖ+(αγ)ᵢₖ+(αβγ)ᵢⱼₖ+εᵢⱼₖₗ

where μ is the overall mean, αᵢ is the effect of the j subject, βᵢ is the treatment effect (CCK-8 or placebo), γₖ is the effect of the meal type (B, L, D), (αβ)ᵢⱼ, (βγ)ⱼₖ, (αγ)ᵢₖ and (αβγ)ᵢⱼₖ are the interaction terms and εᵢⱼₖₗ is the residual or error term. No period or carry over term was assumed. The anovan procedure of MATLAB (The Math Works, Inc.) was used to calculate the ANOVA table where the numbers were not the same numbers of cases for each combination of model and factor (unbalanced).

In addition, daily food consumption for each subject was calculated and expressed as a percentage of consumption reduction. To compare the difference of the mean (and median) both the parametric paired t-test and the non-parametric Wilcoxon rank test were used.

Data were analyzed for each individual and for the group. Analyses were performed for all three measured end points: (1) amount of food consumed (calories), (2) amount of food consumed (grams) and (3) amount taken to plate (grams).

### Results

The effect of CCK-8 administered in accordance with the teachings of the present invention on the calories consumed is shown in Table III from which is seen that intraduodenal administration of CCK-8 produced a statistically significant reduction in the amount of calories consumed (p=0.0128).

TABLE III
The effect of the CCK-8 administration of the calories consumed

| source | Sum Sq. | d.f. | Mean Sq. | F | Prob > F |
|---|---|---|---|---|---|
| αᵢ | 1898371.2 | 5 | 379674.2 | 22.01 | 0 |
| βᵢ | 3523282.3 | 2 | 1761641.2 | 102.12 | 0 |
| γₖ | 110867 | 1 | 110867 | 6.43 | 0.0128 |
| (αβ)ᵢⱼ | 727885.5 | 10 | 72788.5 | 4.22 | 0.0001 |
| (βγ)ⱼₖ | 134146.3 | 5 | 26829.3 | 1.56 | 0.1799 |
| (αγ)ᵢₖ | 31845.3 | 2 | 15922.7 | 0.92 | 0.4007 |
| (αβγ)ᵢⱼₖ | 112107.7 | 10 | 11210.8 | 0.65 | 0.7676 |
| εᵢⱼₖₗ | 1690578.2 | 98 | 17250.8 | | |
| Total | 9106845.5 | 133 | | | |

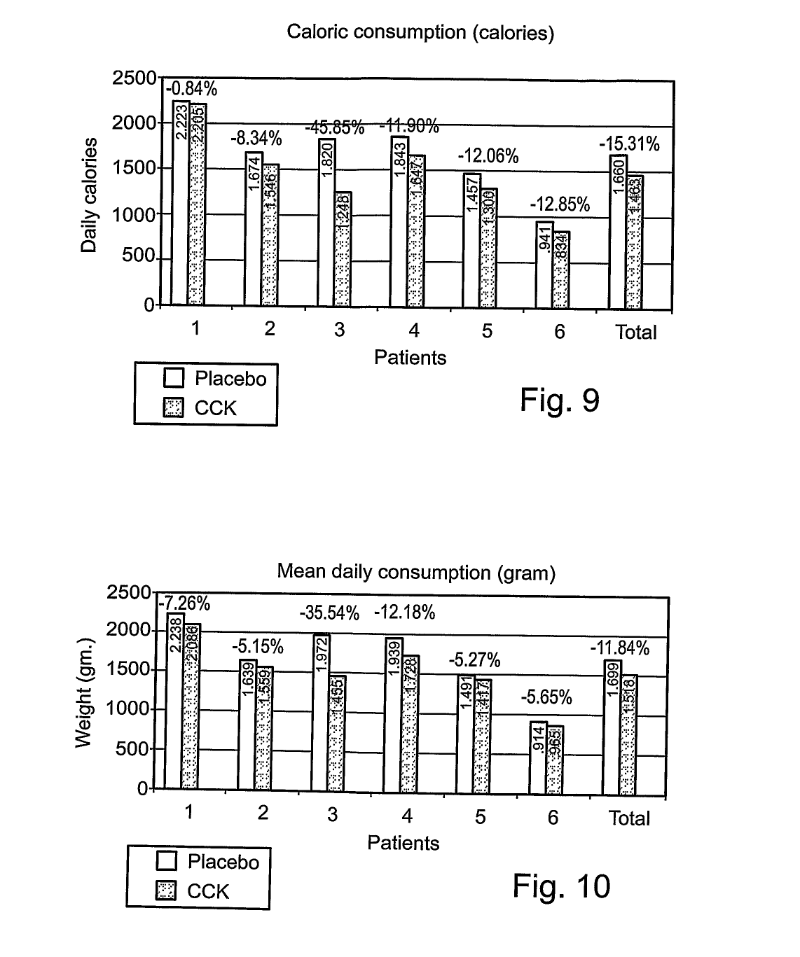

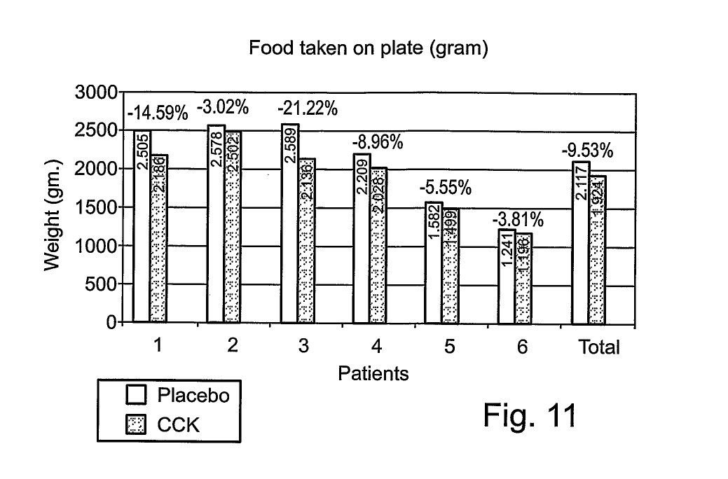

The mean and median amounts of calories consumed by each subject are shown in FIG. 9 and in Table IV from which is seen that the mean decrease in food consumption as expressed in calories was 15.31±14.25% (p=0.03).

TABLE IV
Mean and Median amounts of calories consumed

| | Mean daily consumption | | Median daily consumption | |
|---|---|---|---|---|
| | (calories) | | (calories) | |
| Patient | placebo | CCK-8 | placebo | CCK-8 |
| 1 | 2223 | 2205 | 2223 | 2219 |
| 2 | 1674 | 1546 | 1667 | 1546 |
| 3 | 1820 | 1248 | 1765 | 1198 |
| 4 | 1843 | 1647 | 1810 | 1576 |
| 5 | 1457 | 1300 | 1401 | 1331 |
| 6 | 941 | 834 | 878 | 875 |
| Total | 1660 | 1463 | 1624 | 1457 |
| p-value | 0.06 | | 0.12 | |

The effect of CCK-8 administered in accordance with the teachings of the present invention on the meal size consumed is shown in Table V from which is seen that intraduodenal administration of CCK-8 led to a statistically significant reduction in the amount of food consumed (p=0.0164).

TABLE V
The effect of the CCK-8 administration of food consumed in grams

| source | Sum Sq. | d.f. | Mean Sq. | F | Prob > F |
|---|---|---|---|---|---|
| αᵢ | 1978287.6 | 5 | 395657.5 | 25.24 | 0 |
| βᵢ | 1220448.0 | 2 | 610224.0 | 38.93 | 0 |
| γₖ | 93462.4 | 1 | 93462.4 | 5.96 | 0.0164 |
| (αβ)ᵢⱼ | 839348.5 | 40 | 83934.9 | 5.36 | 0 |
| (βγ)ⱼₖ | 111796.1 | 5 | 22359.2 | 1.43 | 0.2214 |
| (αγ)ᵢₖ | 42419.5 | 2 | 21209.7 | 1.35 | 0.2632 |
| (αβγ)ᵢⱼₖ | 99902.2 | 10 | 9990.2 | 0.64 | 0.7785 |
| εᵢⱼₖₗ | 1535950.1 | 98 | 15673.0 | | |
| Total | 6105394.9 | 133 | | | |

The effect mean and median amounts of grams of food consumed by each subject are shown in FIG. 10 and in Table VI from which is seen that the mean decrease in food consumption as expressed in grams was 11.84±10.87% (p=0.03).

TABLE VI
Mean and Median amounts of calories consumed

| | Mean daily consumption | | Median daily consumption | |
|---|---|---|---|---|
| | (grams) | | (grams) | |
| Patient | placebo | CCK-8 | placebo | CCK-8 |
| 1 | 2238 | 2086 | 2238 | 2134 |
| 2 | 1639 | 1559 | 1659 | 1559 |
| 3 | 1972 | 1455 | 2030 | 1350 |
| 4 | 1939 | 1728 | 2005 | 1763 |
| 5 | 1491 | 1417 | 1495 | 1453 |
| 6 | 914 | 865 | 951 | 866 |
| Total | 1699 | 1518 | 1730 | 1521 |
| p-value | 0.05 | | 0.09 | |

The effect of CCK-8 administered in accordance with the teachings of the present invention on the amount of food taken is shown in Table VII from which is seen that intraduodenal administration of CCK-8 led to a statistically significant reduction in the amount of food consumed (p=0.0219).

TABLE VII
The effect of the CCK-8 administration on food taken in grams

| source | Sum Sq. | d.f. | Mean Sq. | F | Prob > F |
|---|---|---|---|---|---|
| αᵢ | 2868450.0 | 5 | 573690 | 29.11 | 0 |
| βᵢ | 1940146.9 | 2 | 970073.4 | 49.22 | 0 |
| γₖ | 106947 | 1 | 106947 | 5.43 | 0.0219 |
| (αβ)ᵢⱼ | 935578.5 | 10 | 93557.9 | 4.75 | 0 |
| (βγ)ⱼₖ | 85368.2 | 5 | 17073.6 | 0.87 | 0.5067 |
| (αγ)ᵢₖ | 54916.6 | 2 | 27458.3 | 1.39 | 0.2531 |
| (αβγ)ᵢⱼₖ | 211799.9 | 10 | 21180 | 1.07 | 0.389 |
| εᵢⱼₖₗ | 1931379.7 | 98 | 19708.0 | | |
| Total | 8674180.0 | 133 | | | |

The effect on the mean and median amounts of food taken by each of the six subjects are shown in FIG. 11 and in Table VIII from which is seen that the mean decrease in food taken as expressed in grams was 9.53±6.5% (p=0.03).

TABLE VIII
Mean and Median amounts of food taken (grams)

| | |
|---|---|
| Mean daily food taken | Median daily food taken |
| (grams) | (grams) |

| Patient | placebo | CCK-8 | placebo | CCK-8 |
|---|---|---|---|---|
| 1 | 2505 | 2186 | 2505 | 2214 |
| 2 | 2578 | 2502 | 2637 | 2502 |
| 3 | 2589 | 2136 | 2645 | 1991 |
| 4 | 2209 | 2028 | 2259 | 2074 |
| 5 | 1582 | 1499 | 1580 | 1522 |
| 6 | 1241 | 1196 | 1198 | 1172 |
| Total | 2117 | 1924 | 2137 | 1912 |
| p-value | 0.03 | | 0.06 | |

### Summary of Results

As seen from the results presented above and in FIGS. 9, 10 and 11 and summarized in Table IX, administration of CCK-8 in accordance with the teachings of the present invention led to a significant decrease in the amount of calories consumed, the amount of food consumed and the amount of food taken, even when an unlimited amount of food was available. Administration of CCK-8 in accordance with the teachings of the present invention had no noticeable effect on the VAS scores.

TABLE IX
Summary of influence of CCK-8 administration

| patient | Calories consumed % | Food consumed % | Food taken % |
|---|---|---|---|
| 1 | 0.83 | 7.26 | 14.59 |
| 2 | 8.31 | 5.15 | 3.02 |
| 3 | 45.83 | 35.54 | 21.22 |
| 4 | 11.90 | 12.18 | 8.96 |
| 5 | 12.06 | 5.27 | 5.55 |
| 6 | 12.83 | 5.65 | 3.81 |
| Average | 15.89 | 11.84 | 9.53 |

In conclusion, it was observed that CCK-8 administered in accordance with the teachings of the present invention led to a consistent and stable reduction in caloric consumption, consumption of food (in grams) and amount of food placed on a plate despite the food being freely available in buffet setting. The volunteers did not report any CCK-8 induced nausea, vomiting or abdominal pain. It is concluded that the administration of CCK-8 in accordance with the teachings of the present invention leads to a feeling of satiety. The statistically significant average reduction of about 16% in daily caloric intake corresponds to a potential average weight reduction rate of approximately 1 kg month⁻¹. Such a significant weight reduction is associated with improvement in various clinical parameters including reduction of blood glucose levels, reduction of blood pressure and correction of lipid derangements.

Thus, with the study reported above the utility of the gastrointestinal administration of a pharmaceutical composition including a peptide that is a CCK analogue for treating conditions relating to food intake such as obesity, bulimia, eating disorders, overeating, diabetes-related obesity and metabolic syndrome was demonstrated. Thus, the teachings of the present invention provide a method of treatment, comprising administering a composition that comprises an active agent (e.g., a satiety agent, e.g. CCK or an analogue thereof or a derivative thereof) as an active agent and a pharmaceutically accepted carrier to a subject in need thereof by spraying the composition in a part of the gastrointestinal tract of the subject so that the active agent interacts with the luminal wall of the gastrointestinal tract thereby causing a beneficial effect, for example a reduction of calories consumed and/or a reduction of the amount food consumed. In some embodiments the luminal wall is the luminal wall of the duodenum of the subject where CCKa receptors are located.

The study also teaches the use of satiety agents, such as CCK or an analogue thereof or a derivate thereof (e.g., CCK-8) for the manufacture of a pharmaceutical composition (that is to say, a medicament) for use in the treatment of a subject by gastrointestinal administration. In some embodiments, the treatment comprises reduction of calorie intake by the subject and or reduction of the amount of food consumed by a subject, for example in a subject that suffers from a disorder such as obesity, bulimia, eating disorders, overeating, diabetes-related obesity and metabolic syndrome. In some embodiments, the composition is a sprayable composition. In some embodiments, the gastrointestinal administration is duodenal administration.

More generally, it was demonstrated that gastrointestinal administration of a composition including a peptide active agent for treating conditions is possible. Thus, the teachings of the present invention provided a method of treatment, comprising administering a composition that comprises a peptide active agent (such as a hormone) and a pharmaceutically accepted carrier to a subject in need thereof by spraying the composition in a part of the gastrointestinal tract of the subject so that the active agent interacts with the luminal wall of the gastrointestinal tract thereby causes a beneficial effect. In some embodiments the luminal wall is the luminal wall of the duodenum of the subject.

The study also teaches the use of peptides as an active agent for the manufacture of a pharmaceutical composition (that is to say, a medicament) for use in the treatment of a subject by gastrointestinal administration. In some embodiments, the composition is a sprayable composition. In some embodiments, the gastrointestinal administration is duodenal administration.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A device for administering a sprayable composition including an active agent to a luminal wall of the gastrointestinal tract of a subject, comprising:
a) a sprayer configured for deployment in a gastrointestinal tract;
b) a pressure generator configured to dispense a dose of sprayable composition out through said sprayer as a spray upon actuation; and
c) an actuator for actuating said pressure generator upon being triggered.

2. The device of claim 1, configured so that said sprayer is deployable at a specific location in a gastrointestinal tract.

3. The device of claim 1, further comprising:
d) a composition reservoir functionally associated with said pressure generator so that said pressure generator is configured to force sprayable composition from said reservoir out through said sprayer as a spray upon said actuation.

4.-35. (canceled)

36. The device of claim 3, further comprising a composition including a satiety factor or an analogue thereof or a derivative thereof and a pharmaceutically acceptable carrier held in said reservoir.

37. The device of claim 36, wherein said satiety factor is CCK or an analogue thereof or a derivative thereof.

38. The device of claim 3, further comprising a composition including a peptide active agent and a pharmaceutically acceptable carrier held in said reservoir.

39. The device of claim 38, wherein said active agent is a peptide hormone, an analogue thereof or a derivative thereof.

40. The device of claim 39, wherein said active agent is CCK-8.

41. The device of claim 1, further comprising:
e) an anchor, configured to anchor said sprayer at a specific location in the gastrointestinal tract.

42. The device of claim 1, further comprising:
f) an event detector functionally associated with said actuator, configured so that as a result of detection of an event of significance for administration of an active agent, said event detector triggers said actuator.

43. The device of claim 42, wherein said event is a physiological change.

44. The device of claim 1, wherein said sprayer is configured for deployment in the duodenum

45. The device of claim 44, further comprising:
b) a feeder tube including a proximal end functionally associated with said sprayer and a distal end, configured to define a conduit for a sprayable composition from said distal end to said sprayer; and
c) an anchor functionally associated with said distal end of said feeder tube wherein said anchor and said feeder tube are configured so that when properly deployed in the body of a subject, said feeder tube passes through the pyloric sphincter of said subject to maintain said sprayer in the duodenum of a subject.

46. The device of claim 45, further comprising an increased-diameter feature near said distal end of said feeder tube, configured to assist in preventing said sprayer from passing through a pyloric sphincter into the stomach of a subject when properly deployed.

47. The device of claim 45, wherein said feeder tube and said anchoring component are configured so that when properly deployed in the body of a subject, said feeder tube passes through the pyloric sphincter of said subject to maintain said sprayer in the superior portion of the duodenum of a subject.

48. A method of administering an active agent, comprising:
a) deploying a sprayer of a device of claim 1 in a specific location of a gastrointestinal tract of a subject suffering from a condition;
b) providing a sprayable composition comprising an active agent and a pharmaceutically acceptable carrier, said active agent effective in treating said condition; and
c) when necessary, spraying a dose of said sprayable composition through said sprayer against a portion of a luminal wall of said gastrointestinal tract of said subject
thereby administering said active agent so as to treat said condition.

49. The method of claim 48 wherein said necessity of said administering said dose of said active agent is determined by detection of an event of significance for administration of said active agent.

50. The method of claim 49, wherein said event comprises an event selected from the group consisting of gastrointestinal tract activity, food ingestion and hunger, anginal attack, arrhythmia, an asthma attack and an allergic reaction.

51. The method of claim 48, wherein said portion of a luminal wall is a portion of the luminal wall of the duodenum of said subject.

52. The method of claim 48, wherein said active agent is a satiety factor or an analogue thereof or a derivative thereof.

53. The method of claim 52, wherein said satiety factor is CCK or an analogue thereof or a derivative thereof.

54. The method of claim 48, wherein said active agent is a peptide active agent.

55. The method of claim 48, wherein said active agent is a peptide hormone, an analogue thereof or a derivative thereof.

56. The method of claim 55, wherein said active agent is CCK-8.
